(19) United States
(12) Patent Application Publication
(10) Pub. No.: US 2025/0229017 A1
(43) Pub. Date: Jul. 17, 2025

(54) SYSTEM AND METHODS AND PORTABLE BASE FOR BOWEL IRRIGATION

(71) Applicant: Coloplast A/S, Humlebaek (DK)

(72) Inventors: Niels-Frederik Keiser-Nielsen, Nivaa (DK); Nina Hoegh Jensen, Copenhagen N (DK); Stephanie Knoedler, Nivaa (DK); Bo Radmer, Hilleroed (DK)

(21) Appl. No.: 18/853,439

(22) PCT Filed: Mar. 29, 2023
(86) PCT No.: PCT/DK2023/050062

§ 371 Date: Oct. 2, 2024

(30) Foreign Application Priority Data
Apr. 8, 2022 (DK) PA 2022 70197
Nov. 17, 2022 (DK) PA 2022 70565

(51) Int. Cl. A61M 3/02

(52) U.S. Cl. CPC A61M 3/0266; A61M 3/0258; A61M 2207/00; A61M 2210/1064

(57) ABSTRACT

A portable base for a system for irrigation of the bowels of a user is provided. The portable base comprises a canister unit for an irrigation liquid, and a lid unit for engaging with the canister unit. In particular, the lid unit of the portable base comprises a rim portion comprising an annular skirt that is movably arranged around the rim portion. The portable base is adapted to be shifted to a locked configuration by rotating the annular skirt. The arrangement of the annular skirt around the rim portion facilitates easier shifting to the locked configuration because less torque is required to be applied by the user for the locking/scaling. A system for irrigating the bowels of a user and a method of sealing the system are also provided.

A portable base for a system for irrigation of the bowels of a user as well as a system and a method of bowel irrigation is provided. The portable base comprises a canister unit for an irrigation liquid, and a lid unit for engaging with the canister unit. In particular, the lid unit of the portable base has an annular rim portion with an annular skirt that is rotatably arranged around the rim portion. The portable base is adapted to be shifted between an unlocked (open and unsealed) configuration and a locked (closed and sealed) configuration (and vice versa) by rotating the skirt. The arrangement of the annular skirt on the rim portion facilitates easier shifting between the unlocked and locked configuration, and vice versa, because less torque is required to be applied by the user for sealing/unsealing of the portable base.

### BACKGROUND

Control of voluntary bowel functions is frequently limited or absent in patients suffering from certain disabilities, such as spinal injuries, multiple sclerosis, or spina bifida. Such lack of control of voluntary bowel functions typically results in faecal incontinence or intractable constipation, as patients have significantly reduced ability to sense the presence of faeces in the colon terminal part and the rectum and to sense the evacuation stimulus.

It is known to accomplish bowel emptying by irrigation (i.e. flushing) of the bowels via the rectum, by an irrigating fluid, such as tap water or saline, which is provided through an intermittent catheter with a tip which is configured and sized for insertion into the rectum (the rectum being the terminal section of the alimentary canal; from the sigmoid flexure to the anus), where it remains in a fixed position and held by an inflatable element, which may be inflatable by air or by water with a pump. Once the rectum or stoma has been flushed with the irrigating liquid, the inflatable element is removed, and the liquid and faeces can evacuate. Some known irrigation systems suffer from being bulky, complicated, and difficult to handle and to clean properly after use. Moreover, some users of irrigation systems have limited dexterity and/or finger strength due to medical conditions and therefore may have difficulty in handling the components of the irrigation system, including particularly the opening, and closing of the container in the process of cleaning the water container and/or in the process of filling (or emptying) irrigation liquid from the container.

Thus, users of irrigation systems and health care professionals alike would welcome improvements in components of bowel irrigation systems.

### SUMMARY

In one aspect, the present disclosure provides a portable base for a bowel irrigation system. In another aspect, the disclosure relates to a bowel irrigation system including a portable base as described herein. In yet another aspect, the disclosure relates to a method of irrigating the bowels of a user with an irrigation system including the portable base disclosed herein. In yet another aspect, the disclosure relates to a method of sealing a canister unit of a bowel irrigation system disclosed herein. The aspects are further defined by the appended claims.

### BRIEF DESCRIPTION OF THE DRAWINGS

The accompanying drawings are included to provide a further understanding of embodiments and are incorporated in and constitute a part of this specification. The figures illustrate embodiments and together with the description explain principles of embodiments. Other embodiments and many of the intended advantages of all embodiments will be readily appreciated as they become better understood by reference to the following detailed description.

Various exemplary embodiments and details are described hereinafter, with reference to the figures when relevant. It should be noted that the figures may or may not be drawn to scale and that elements of similar structures or functions are represented by like reference numerals throughout the figures. It should also be noted that the figures are only intended to facilitate the description of the embodiments. They are not intended as an exhaustive description of the invention or as a limitation on the scope of the invention. In addition, an illustrated embodiment needs not have all the effects or advantages shown. Conversely, an effect or an advantage described in conjunction with a particular embodiment is not necessarily limited to that embodiment and may be practiced in other embodiments even if not so illustrated, or if not so explicitly described.

The figures include the following:

FIG. 1 is a perspective view illustrating one embodiment of a portable base of a system for irrigating the bowels of a user in a closed/sealed configuration.

FIG. 2 is a cross-sectional view of one embodiment of a portable base in an unlocked and unsealed/open configuration.

FIG. 3A is a schematic perspective view of an annular skirt of a lid unit of the portable base

FIG. 3B is a cross-sectional view of the embodiment of the annular skirt of the portable base according to FIG. 3A.

FIG. 4 is a schematic perspective view illustrating one embodiment of an annular neck portion of a canister unit of the portable base.

FIG. 5A is a schematic sectional perspective view of a portion of a sealing ring to be attached around an annular rim portion of the lid unit of the portable base according to one embodiment.

FIGS. 5B-5C are schematic sectional views of embodiments of a portable base of a system for irrigating the bowels of a user including a sealing ring attached around an annular rim portion of the lid unit.

FIGS. 6A-6C are schematic sketches illustrating sectional views of one embodiment of a sealing arrangement of a portable base in three different positions.

FIGS. 7A-7D are schematic sketches illustrating in cross-section four alternative embodiments of a sealing of a portable base.

FIG. 8 is a schematic, perspective sectional view of one embodiment of an annular neck portion of the canister unit of the portable base.

FIG. 9 is a schematic, perspective sectional view of one embodiment of a lid unit of the portable base and particularly illustrating an annular rim portion thereof.

FIG. 10 is a top view of one embodiment of the portable base with the lid unit positioned on top of the canister unit and with a top of the lid unit removed for illustration purposes.

FIG. 11 is a schematic perspective view of a portion of the lid unit in position on top of the canister unit in one embodiment of the portable base.

FIGS. 12A-12C are each schematic top views illustrating a section of an annular neck portion of the canister unit of the portable base and with an annular skirt of the lid unit of the portable base shown radially outward thereof.

FIG. 13 is an enlarged view of a portion of the annular skirt illustrated in FIG. 3A.

FIG. 14 is a schematic, perspective view illustrating one embodiment of a system for irrigating the bowels of a user including one embodiment of a portable base.

FIG. 15 is a diagram illustrating the steps of a method of irrigating the bowels of a user with a bowel irrigation system incorporating the portable base.

FIG. 16 is a diagram illustrating the steps of a method of sealing a portable base of a system for irrigating the bowels of a user with a bowel irrigation system incorporating the portable base.

### DETAILED DESCRIPTION

In the following Detailed Description, reference is made to the accompanying drawings, which form a part hereof, and in which is shown by way of illustration specific embodiments in which the invention may be practiced. In this regard, directional terminology, such as “top,” “bottom,” “front,” “back,” “leading,” “trailing,” and equivalent, is used with reference to the orientation of the Figure(s) being described. Because components of embodiments can be positioned in a number of different orientations, the directional terminology is used for purposes of illustration and is not necessarily limiting. It is to be understood that other embodiments may be utilized, and structural or logical changes may be made without departing from the scope of the present invention. The following detailed description, therefore, is not to be taken in a limiting sense, and the scope of the present invention is defined by the appended claims.

It is to be understood that individual features of the various exemplary embodiments described herein may be combined with each other, unless specifically noted otherwise.

In this disclosure, solid and liquid wastes from the bowels of a person may be referred to as both “output,” “waste(s),” and “fluids” interchangeably. A person using the irrigation system may be referred to as “patient” or “user”. However, in some contexts, “user” can also relate or refer to a health care professional (HCP), such as a doctor/physician, nurse, or other person.

In this disclosure, ‘axial direction’ should generally be considered as vertical seen in relation to a top and bottom of the portable base. The radial direction should be understood as transverse to the axial direction. In some sentences, the words “inner” and “outer” can be used. These qualifiers should generally be perceived with reference to the radial direction, such that a reference to an “outer” element means that the element is farther away from a centre portion of a component than an element referenced as “inner”. In addition, “innermost” should be interpreted as the portion of a component forming a centre of the component and/or being adjacent to the centre of the component. In analogy, “outermost” should be interpreted as a portion of a component forming an outer edge or outer contour of a component and/or being adjacent to that outer edge or outer contour. In further analogy this is also the case about the use of the words “uppermost” and “lowermost”.

The use of phrases such as (but not limited to) “substantially” and “approximately” as qualifiers of certain features or effects in this disclosure, is intended to mean that any deviations are within tolerances that would normally be expected by the skilled person in the relevant field.

The use of the word “generally” as a qualifier to certain features or effects in this disclosure is intended to mean—for a structural feature: that a majority or major portion of such feature exhibits the characteristic in question, and—for a functional feature or an effect: that most outcomes involving the characteristic provide the effect, but that exceptionally some outcomes may not provide the effect.

The phrase “adjacent” should be interpreted as being “close to and/or in contact with, or temporarily brought into contact with, but not attached to [something].”

In the present disclosure, “portable” is to be understood as being easy to carry or move, so that a “portable” element can be used in different places and can be easily and conveniently transported. Particularly, this should be understood is relation to some types of available irrigation systems for the bowels of a user including components which are large and bulky and intended for bedridden and/or hospitalized users only.

In the present disclosure, “jut” is to be understood as something that bulges out or is protuberant or projects from a form.

In the present disclosure, “fixed” is to be understood as something that is fastened in one position so that it does not move.

In the present disclosure, “section” is to be understood as something that is one of several parts or pieces that fit or join with others to constitute a whole (an entirety of an) object.

In the present disclosure, “complementary” is to be understood such that things that are “complementary” combine or fit well together.

In the present disclosure, “crosspiece” is to be understood as an elongated part that extends across something.

In a first aspect, the present disclosure relates to a portable base for a system for irrigating the bowels of a user, comprising:
a canister unit comprising a volume for an irrigation liquid and an annular neck portion circumscribing an opening around a central vertical axis of the portable base; and
a lid unit configured to be positioned on the annular neck portion and over the opening for closing of the canister unit, the lid unit having an axial extent along the central vertical axis between a top and a bottom of the lid unit, and comprising an annular rim portion and an annular skirt, the annular skirt configured to be partially rotatable relative to the annular rim portion when the lid unit is positioned aright on the annular neck portion;
wherein a radially outward facing surface of the annular neck portion of the canister unit comprises at least one projecting jut;
wherein a radially inward facing surface of the annular skirt of the lid unit comprises at least one thread;
wherein a sealing is provided at the annular rim portion to seal between the canister unit and the lid unit in a locked configuration of the portable base;
wherein the sealing is configured to be moved by a force exerted by the annular neck portion; and
wherein rotating the annular skirt relative to the annular rim portion causes the at least one thread of the annular skirt to engage with the at least one jut of the annular neck portion and thereby move the sealing and lock the lid unit onto the canister unit in the locked configuration of the portable base.

The canister unit includes a volume for an irrigation liquid. The irrigation liquid can be temperate tap water or in some cases a saline physiological solution. The canister unit comprising the volume for the irrigation liquid can be considered a first major or primary component of the portable base. The canister unit includes an annular neck portion circumscribing an opening around a central vertical axis of the portable base. In embodiments, the annular neck portion comprises a vertical flange extending generally parallel to a central vertical axis of the portable base. In embodiments, an upper part (area or zone) of the flange includes a tapering, i.e., is at an angle to the central vertical axis. In embodiments, the annular neck portion transitions into a shoulder portion of the canister unit. The transition between the annular neck portion and the shoulder portion is located at a lower part (area or zone) of the neck portion. In embodiments, the shoulder portion of the canister unit (further or in turn) transitions into a sidewall of the canister unit. The transition between the shoulder portion and the sidewall of the canister unit is located at a lower part (area or zone) of the shoulder portion and can further be located radially outermost on the shoulder portion. In embodiments, the annular neck portion is offset radially inwardly from the sidewall of the canister unit by (or at) the shoulder portion and the annular neck portion is provided radially closer to the central vertical axis than is the sidewall of the canister unit.

In embodiments, the sidewall of the canister unit extends substantially parallel to the central vertical axis. Thus, the canister unit generally takes the shape of a cylinder when viewed in cross-section. In such embodiments, the annular neck portion does not form a portion offset radially inwardly from the sidewall of the canister unit by a shoulder portion. The annular neck portion is thereby provided as an upper portion of the sidewall of the canister unit and is generally at the same distance to the central vertical axis as the remainder of the sidewall of the canister unit.

In embodiments, the sidewall of the canister unit extends generally at an angle to the central vertical axis of the portable base. In embodiments, the canister unit is configured to have a first smaller diameter at a top portion of the canister unit and a relatively larger, second diameter at a bottom portion of the canister unit. The canister unit is adapted to stand by itself on a generally level surface of the surroundings. This is useful for making the portable base in its entirety stable when standing on a surface during use.

In embodiments, the canister unit comprises a non-planar underside (or bottom). In embodiments, the non-planar underside includes an apex that coincides with the central vertical axis, the apex being closer to a top portion of the canister unit than any other location on the underside of the canister unit.

The annular neck portion of the canister unit generally circumscribes the opening. The annular neck portion can be understood as delimiting, or defining or surrounding, the extent (or size) of the opening of the canister unit. Particularly, the opening provides a passage which is highly useful when cleaning or disinfecting the canister unit. In embodiments, the opening is relatively large, which greatly assists a user in cleaning the inside volume of the canister unit by inserting a hand or the fingers thereinto for cleaning. The opening can also at least partly provide a passage for filling and emptying irrigation liquid into and from the canister unit. In embodiments, the annular neck portion of the canister unit includes a radially inward facing surface and/or a radially outward facing surface. In embodiments, one or both of the radially inward and outward facing surfaces extend in a direction substantially parallel to the central vertical axis of the portable base.

The portable base includes a lid unit configured for closing of the opening of the canister unit. The lid unit is configured to be positioned on the annular neck portion and over the opening for closing of the canister unit. The lid unit has an axial extent along the central vertical axis of the portable base between a top and a bottom of the lid unit. Thus, the lid unit has a height in a direction parallel to the central vertical axis. The lid unit is provided with an external top surface (‘external’ to be understood as facing towards the surroundings away from the canister unit) and an internal bottom surface (‘internal’ to be understood as facing the volume for the irrigation liquid inside the canister unit). The use of the words ‘external’ and ‘internal’ shall also be understood in relation to the lid unit being configured for attachment to (or engagement with) the canister unit over the annular opening (to close the canister unit). In embodiments, the axial extent of the lid unit measured along the central vertical axis is in a range of 25 mm-60 mm. In a specific implementation, the axial extent is generally 35 mm. Individual portions or elements having a greater axial extent measured along the central vertical axis can extend longer from the top and/or bottom surface of the lid unit, but the top and bottom surfaces can be provided at generally 35 mm.

The lid unit includes an annular rim portion and an annular skirt, and the annular skirt is configured to be partially rotatable relative to the annular rim portion when the lid unit is positioned aright on the annular neck portion. The annular rim portion is formed at a lower portion of a radially outward facing lateral wall of the lid unit. The lid unit has a first diameter at the annular rim portion. The radially outward facing lateral wall of the lid unit also includes an upper portion. The lid unit has a second diameter at the upper portion of the lateral wall, the second diameter being greater than the first diameter at the annular rim portion. Thus, it is understood that the annular rim portion of the lid unit is offset radially inward in relation to the upper portion of the lateral wall of the lid unit when viewed in relation to the central vertical axis. The phrases “upper” and “lower” should be understood in relation to positions on the central vertical axis. In embodiments, the lateral wall of the lid unit, or at least the annular rim portion, is provided parallel to the central vertical axis. In embodiments, the lateral wall of the lid unit defines or includes a shoulder at a transition between the lower portion and the upper portion, with the lower portion provided by the annular rim portion. In embodiments, the shoulder is located at a transition between the upper portion of the lateral wall of the lid unit and the annular rim portion. In embodiments, the shoulder provides a downwardly facing surface. In embodiments, the shoulder forms the transition between the upper portion of the lateral wall of the lid unit and the annular rim portion of the lid unit. In embodiments, the shoulder is provided to extend annularly entirely along the upper portion (full circle of the lid unit). In embodiments, a radial extent of the shoulder in a direction perpendicular to the central vertical axis can be configured within a range of 5-30 mm (measuring along the downwardly facing surface).

The annular skirt can include a peripheral wall extending 360 degrees around the lid unit. The peripheral wall of the annular skirt forms a radially inward facing surface and a radially outward facing surface of the annular skirt. The annular skirt is provided such that is extends around the annular rim portion of the lid unit, i.e. with the radially inward facing surface generally facing towards the radially outward facing surface of the annular rim portion. In embodiments, the annular skirt is formed with a third diameter being equal to the second diameter of the lid unit at the upper portion of the lateral wall. Thereby, the radially outward facing surface of the annular skirt aligns laterally with the upper portion of the lateral wall.

That the annular skirt is configured to be partially rotatable is intended to mean that the annular skirt can be rotated (or turned/moved) partway around and relative to the annular rim portion, but not a “full circle”. Moreover, the wording “when the lid unit is positioned aright on the annular neck portion” means that the lid unit must be in a correct, predetermined position on the annular neck portion of the canister unit for the annular skirt to be at least partially rotatable relative to the rim portion. The lid unit does not allow rotation of the annular skirt if the lid unit is not positioned aright on the canister unit, and thus, without aright positioning, the annular skirt cannot (neither partially nor fully) be rotated relative to the rim portion. Note that when the lid unit is placed on the canister unit over the opening, but without being positioned “aright” on the canister unit, the lid unit in its entirety is easily (with only negligible resistance from friction) rotatable relative to the canister unit. This provides for easy locating of the lid unit onto the canister unit for the user who can simply and easily rotate the entire lid unit (in both clockwise and counterclockwise direction) until it reaches the correct positioning, in which position only the partial rotation of the annular skirt relative to the rim portion will be possible.

The annular skirt includes a radially inward facing surface. The radially inward facing surface of the lid unit includes at least one thread. In embodiments, the radially inward facing surface is provided by the peripheral wall of the annular skirt. In embodiments, the peripheral wall of the annular skirt includes the at least one thread. In embodiments, the at least one thread extends in a direction parallel to a plane which is generally perpendicular to the central vertical axis. In embodiments, the thread of the peripheral wall of the annular skirt extends radially inward towards the central vertical axis. In embodiments, the thread of the peripheral wall is configured to engage with and slide in the annular neck portion of the canister unit. In embodiments, the thread of the peripheral wall is configured to engage with and slide in a groove or track of the canister unit. In embodiments, the groove or track is provided in the annular neck portion of the canister unit. In embodiments, the thread of the peripheral wall of the annular skirt is formed as a flange or flange-like element. In embodiments, the radially inward facing surface provided by the peripheral wall of the lid unit includes two or more threads or thread portions, such as three, such as four, such as five, such as six, such as seven, such as eight, such as nine, such as ten or even further threads or thread portions.

In embodiments, the radially outward facing surface of the annular neck portion of the canister unit includes at least one projecting jut. In embodiments, the radially outward facing surface of the annular neck portion includes two projecting nuts. In embodiments, the radially outward facing surface of the annular neck portion includes three projecting nuts. In embodiments, the radially outward facing surface of the annular neck portion includes four projecting nuts. In embodiments, the radially outward facing surface of the annular neck portion includes more than four projecting nuts. In embodiments, the projecting nuts are provided equidistantly on the annular neck portion. In embodiments, the projecting nuts are provided in groups of two to four nuts located immediately adjacent to each other, the groups being provided equidistantly on the annular neck portion.

In embodiments, the at least one thread of the peripheral wall of the annular skirt is configured to cooperate with the at least one projecting jut on the radially outward facing surface of the annular neck portion of the canister unit. In embodiments, the at least one thread and the at least one projecting jut are configured to provide complementary engagement means. Thus, the at least one thread and the at least one projecting jut are suitable for and configured to go into engagement with each other to attach the lid unit to the canister unit in the locked configuration of the portable base.

In embodiments, the radially inward facing surface of the annular skirt formed by the peripheral wall includes at least one radially inward extending support flange. In embodiments, the support flange(s) is/are configured to be located on a ledge of the lid unit in such a manner that the support flange rests on the ledge. The engagement between the support flange and the ledge is further useful in keeping the annular skirt connected to the lid unit, such that the annular skirt cannot be separated from the remainder of the lid unit. Other elements of the lid unit can assist in maintaining the annular skirt connected thereto. In addition, the engagement between the support flange of the annular skirt and the ledge of the lid unit provides for the support flange to slide on the ledge and help provide for the at least partial rotation of the annular skirt relative to the annular rim portion of the lid unit. In embodiments, the ledge of the lid unit includes an annular ledge, extending entirely full circle around the lid unit. In embodiment, the ledge of the lid unit includes two or more separate ledge segments. In embodiments, the ledge of the lid unit is located at the annular rim portion of the lid unit. In embodiments, the ledge of the lid unit extends radially outward from the annular rim portion of the lid unit. In embodiments, the support flange can be understood to help provide each of an attaching function, a movement controlling function, and a sliding function, or capability, of the annular skirt in relation to the lid unit (the support flange being engaged and held “in place”, at least by the ledge of the lid unit and optionally also by other elements of the lid unit. The engagement between the support flange of the annular skirt and the ledge of the lid unit provided at the rim portion, allows the annular skirt to rotate around the central vertical axis and/or prevents the annular skirt from disengaging from the lid unit.

A sealing is provided at the annular rim portion to seal between the canister unit and the lid unit in a locked configuration of the portable base. Thus, it is understood that in the locked configuration of the portable base the lid unit is locked onto the canister unit and the sealing is engaged to seal the lid unit and the canister unit with each other.

The sealing is configured to be moved by a force exerted by the canister unit, such as by the annular neck portion of the canister unit. In embodiments, the annular neck portion of the canister unit exerts the force on the sealing when the user rotates the annular skirt relative to the rim portion. The rotation of the annular skirt causes the at least one thread of the annular skirt to engage with the at least one jut of the annular neck portion. This rotational movement of the annular skirt urges the entire lid unit to move axially downward towards the canister unit in a direction parallel to the central vertical axis. The resulting downward movement of the lid unit thereby causes the neck portion of the canister unit to engage with and move the sealing. As a simultaneously occurring effect, the sealing is pressed (squeezed) against the annular neck portion of the canister unit. Thus, the lid unit engages with the canister unit to seal and lock them together in the locked configuration of the portable base.

In embodiments, the sealing is attached to the annular rim portion of the lid unit and brought into an initial abutment with the annular neck portion of the canister unit when the lid unit is positioned aright on the annular neck portion. A seal (sealing effect) is effectuated when the sealing is urged or pressed into engagement with the annular neck portion by rotation of the annular skirt to cause the complementary engagement means, e.g., the jut(s) and thread(s), to engage with each other and shift the portable base to the locked configuration. During the sealing (locking) action, the engagement of the complementary engagement means with each other, and the force incurred on the components when the user rotates the annular skirt, results in an axially downward movement of the lid unit in relation to the canister unit, which in turn causes the sealing to be pressed into leak-tight engagement with the annular neck portion and creates a seal (sealing effect) between the lid unit and the canister unit. The rotation of the annular skirt and the resulting axially downward movement of the lid unit leads to pressure being applied to the material of the sealing. One advantageous effect of the provision of a sealing according to the disclosure is that irrigation liquid in the volume of the canister unit of the portable base can be pressurized, and maintained pressurized, independently of tubing of a system for irrigation the bowels of a user.

By rotating the annular skirt, the at least one thread of the peripheral wall of the annular skirt is adapted to come into engagement with the at least one projecting jut on the radially outward facing surface of the annular neck portion, which rotation results in the securing of the lid unit onto the canister unit and shifting of the portable base to the locked configuration. In embodiments, the at least one thread of the peripheral wall of the annular skirt is adapted to form a “male” part of the complementary engagement means. In embodiments, the at least one projecting jut on the radially outward facing surface of the annular neck portion is adapted to form a “female” part of the complementary engagement means. Other implementations of complementary engagement means are acceptable.

In embodiments, wherein the peripheral wall of the annular skirt includes more than one individual thread or thread segment, the threads or thread segments can be adapted to complementary engage with a corresponding number of projecting juts on the annular neck portion of the canister unit. In embodiments, the one or more threads or thread segment(s) on the annular skirt is/are sloped or tapered such that by rotational movement of the annular skirt including the thread or thread segment(s) relative to the complementary jut or juts on the neck portion of the canister unit, the lid unit is moved in an axial direction parallel to the central vertical axis (sealing and locking action as described above). In embodiments, the at least one jut on the neck portion of the canister unit is also sloped or tapered. In embodiments, a sloping or tapering of the at least one thread of the annular skirt is configured to be complementary to a sloping or tapering of the at least one jut of the canister unit to provide for an easy, sliding engagement of the two elements. In other embodiments, the thread(s) and jut(s) can be configured to prevent easy bypassing of each other.

Embodiments of the disclosure are advantageous in that the way in which the lid unit and the canister unit are put together and sealed/locked, is eased, because it is not required to carefully and/or precisely align the annular skirt of the lid unit and the neck portion of canister unit in a specific position relative to each other to rotate the skirt. Instead, the elements provide that the complementary engagement means correctly “slide into position” relative to each other before the portable base can be sealed/locked by the partial, rotational movement of the annular skirt. According to the invention, skewed or otherwise incorrect alignment of the complementary engagement means and thus insufficient engagement and/or sealing between the canister unit and the lid unit is prevented.

A particular advantageous effect of embodiments of the above-described structure of a portable base is that, for closing and opening of the canister unit with the lid unit, it is not necessary to overcome a frictional force required to rotate the sealing. When the lid unit is located on the canister unit, but not yet positioned aright thereon, the lid unit can be rotated without any portion of the sealing being in frictional engagement with the canister unit. Thus, it will be very easy to rotate the lid unit sitting on the canister unit until it reaches its aright (correct) position in which it becomes possible for the annular skirt to be rotated relative to the annular rim portion. Moreover, when the portable base is not in its locked configuration, there is no, or only very little (negligible), frictional contact or engagement between the sealing provided on the lid unit and the canister unit. A further advantage is that the sealing itself does not have to be moved (rotated) annularly around the central vertical axis during rotation of the annular skirt of the lid unit. Instead, the sealing only needs to move a relatively short distance in radial and axial directions by the force incurred by the user rotating the annular skirt of the lid unit. Thus, according to the disclosure, a portable base for a system for irrigating the bowels of a user is provided, wherein the force needed to seal and unseal (close and open) the container (canister unit) containing a volume of irrigation liquid is significantly reduced, in that it is not necessary for the user to overcome a combination of both a frictional force for rotating the sealing itself and a normal force required to move the sealing in a vertical (axial) direction parallel to the central vertical axis. This effect is particularly useful as it allows for easier unlatching or unfastening of the lid unit from the canister unit in its entirety. It also allows for the provision of an opening for an irrigation liquid container which can be of relatively large diameter or size thereby making it easier for the user to clean the inside of the container volume and/or potentially easier to fill the container with irrigation liquid for use. Making cleaning of the components of the portable base easier, particularly cleaning of the canister unit, holding the irrigation liquid, by allowing the user to get a hand inside the canister unit during the cleaning procedure, is useful and advantageous and provides a solution that is both convenient and intuitive for the user. Moreover, lowering or reducing the necessary force required to open and close the container is also advantageous and useful in that users of irrigation systems often lack hand dexterity due to various medical conditions and/or plainly lack sufficient physical strength. Additionally, the mere reason that some users have a relatively small hand size, can make it difficult to rotate closures or lids on containers having relatively large openings/lids. According to the present invention, the elimination of the need to overcome a frictional force required for rotation of a sealing in an irrigation liquid container, alleviates, or at least reduces, each of these issues and helps provide the option of providing a container having a large opening, which in turn helps provide for the easy cleaning solution.

It is thus to be understood that a portable base for a system for irrigating the bowels of a user is provided, in which closing and opening and particularly sealing of the container for holding an irrigation liquid is more easily done by virtue of the annular skirt being rotatable around the annular rim portion of the lid unit. According to the disclosure, any need for simultaneously forcing the sealing to be rotated for closing and opening “alongside” with other elements of the lid unit, can be avoided. The construction facilitates that only a slight radial and axial movement of the sealing takes place instead of having to rotate the sealing entirely for the opening/closing (and sealing/unsealing) action to occur. In addition, the slight movement of the sealing by rotating the annular skirt allows for the internal air-pressure in the portable base to be relieved (released or equalized) already prior to the lid unit being entirely freed from engagement from the canister unit. The achieved result of the slight radial and axial movement of the sealing, and the “early” relief or “initial” equalization of pressure in the canister unit, these taking place at slightly different moments in time, additionally helps reduce or eliminate a frictional force required to remove the lid unit from the canister unit (i.e., “finally” disengaging the complementary engagement means from each other becomes easier). It is to be understood, that the portable base shifts from a sealed (and locked) to an unsealed (and unlocked) configuration because of the rotation of annular skirt, the rotational movement causing (leading to) a slight radial and axial movement of the sealing, but not a rotation of the sealing. After the opening action, and relief of pressure in the canister unit, any force required for the subsequent removal of the lid unit from the canister unit is negligible, and thus much easier overcome by users. Additionally, the slight radial and axial movement of the sealing can advantageously be assisted even further by adapting (or customizing) the size, shape, and/or form of the complementary engagement means, particularly the thread(s) or thread segment(s) of the peripheral wall of the annular skirt, for example, by way of a particular tapering or sloping of this/these.

It is an additional advantage of the present disclosure that no or only little rotational movement or turning of the entire lid unit is needed to unseal/open and/or seal/close the canister unit with the lid unit. Firstly, in analogy to above, avoiding rotation of the lid unit in its entirety means that less force is required by the user for opening and/or closing of the container containing the irrigation liquid. Secondly, avoiding rotation of the lid unit is advantageous in embodiments of an irrigation system, wherein one or more components of the system are incorporated into the lid unit (for example control and power components and/or a pump unit) and further in embodiments wherein tubing for the irrigation liquid is attached to the portable base via (in engagement with) the lid unit. In such cases, the portable base including the lid unit and the canister unit makes handling of the different components of an irrigation system in relation to each other easier and less cumbersome. As an example, potential unintended twisting and/or winding of irrigation liquid tubing extending from the lid unit, caused by rotation of the lid during the opening and closing of the portable base can be avoided, thus reducing the potential risk of malfunction and/or of impeding of liquid flow in the tubing. Among other advantages, the lid unit can additionally house one or more delicate control components in one or more internal chambers, e.g., a CPU and/or other electronics, whereby it is useful to avoid movements of the entire lid unit to reduce unnecessary shifting and stress on the housed components from externally incurred rotation forces. The option of attaching the lid unit on the canister unit and avoiding any substantial rotation of the entire lid unit itself, further adds flexibility for a user of an irrigation system incorporating the portable base in terms of easing requirements for putting together the components of the system in a specific, and/or, predetermined order. Thus, in embodiments according to the disclosure it does not matter if, for example, the irrigation liquid tubing has been inadvertently attached to the connection on the lid unit before closing/sealing of the lid unit onto the canister unit, because the lid unit itself with the attached tubing is not rotated during the closing/sealing action, only the annular skirt is partially rotated or turned to close and seal the portable base.

In embodiments, the sealing includes at least a first portion fixed to the lid unit and a second portion configured to engage with the canister unit and a connecting portion extending between the first portion and the second portion. In embodiments, the first portion is fixed to the annular rim portion of the lid unit and the second portion is configured to engage with the annular neck portion of the canister unit and the force exerted by the annular neck portion is exerted on the second portion of the sealing. In embodiments, at least the first portion of the sealing is fixed to the rim portion by a polymer-based adhesive material. Alternatively, or additionally, in embodiments, at least the first portion of the sealing can be mechanically fastened or attached to the rim portion of the lid unit. In embodiments, the sealing includes a rubber-based or silicone-based material (or other suitable polymeric material). In embodiments, the sealing is suitably made from a specific silicone material. In embodiments, the specific silicone material is configured to have a Shore A hardness in a range of 35-60 Shore A, such as 50 Shore A. In embodiments, the sealing is provided as a separate element in the form of an annular ring. The annular ring can be attached around the annular rim portion of the lid unit. In embodiments, an inner diameter of the annular ring of the sealing can be configured to be slightly less than an outer diameter of the annular rim portion, so that the annular ring can fit tightly around the annular rim portion. In embodiments, the first portion of the sealing includes a radial inner surface including at least one radially inward extending protrusion adapted to be particularly suitable as the sealing's “packing-” and/or sealing surface, i.e. a surface which sits against the annular rim portion of the lid unit. In embodiments, the connecting portion is provided radially outside of the first portion and may extend annularly around the first portion. In embodiments, a material thickness of the sealing material of the connecting portion is less than a thickness of the sealing material of the first portion. In embodiments, the connection portion can have an arched profile when viewed in a cross-sectional view of the sealing. Thereby, the connecting portion can be formed as an arch extending between the first portion and the second portion of the sealing.

In embodiments, the second portion of the sealing includes an abutment section configured for engagement with a top surface of the annular neck portion of the canister unit and a lip section configured to seal against at least a radially inward facing surface and/or a radially outward facing surface of the annular neck portion. In embodiments, the abutment section includes a first flange extending from the connecting portion of the sealing. The first flange can include an at least partially rounded-off end portion. In embodiments, a downwardly facing surface of the first flange of the abutment section of the second portion of the sealing is generally flat or level and adapted to engage with the top surface of the annular neck portion. In embodiments, the lip section includes a second flange extending from the connecting portion of the sealing. In embodiments, the second flange of the lip section extends between the connecting portion and the first flange of the abutment section of the second portion. In embodiments, the first flange of the abutment section extends in a first direction from the connecting portion and the second flange of the lip section extends in a second, different direction from the connection portion. These configurations of the second portion of the sealing provides a sealing in which one part or portion is subjected to and picks up a movement-incurring force and another part or portion provides the sealing effect. This is advantageous in that deformation of the sealing-effect-providing part or portion can be reduced and/or kept isolated from direct application of force from the user's rotation of the annular skirt. Further advantageous effects of these embodiments include the possibility to obtain a relatively high sealing pressure from a relatively low axial force because the sealing can be understood to “roll” into position (i.e. the locked configuration of the portable base) substantially without being influenced by the friction coefficient between the sealing material and the canister unit.

In embodiments, the abutment section comprises a first flange and the lip section comprises an apex of a transition between the second portion and the connecting portion of the sealing.

Further advantageous effects of the various embodiments including a sealing comprising a first portion and a second portion connected by a connecting portion include that the sealing is easy to produce and clean (in use) and that it is easy to dimension the required sealing force. Moreover, these implementations of a sealing have greatly reduced sensitivity to tolerance issues/requirements. This is particularly pronounced with regard to tolerances in the radial direction but also beneficial with regard to tolerances in the axial direction.

In embodiments, the abutment section comprises a first flange and the lip section comprises a second flange and wherein the first flange and the second flange define an angle of at least 30° between each other. In embodiments, the first flange and the second flange define an angle between each other being in a range of 30°-85°, such as 40°, such as 45°, such as 50°, such as 60°, such as 70°, such as 75°, such as 80°, such as 85°. In embodiments, the first flange and the second flange are substantially perpendicular to each other. These embodiments provide and/or further reinforce the advantageous effects of isolating the part of the sealing picking up the force incurred by the rotation of the annular skirt from the part of the sealing providing the sealing effect. The embodiments further provide a range of options for controlling the forces required to move the sealing and to seal against the annular neck portion of the canister unit. Additionally, or alternatively, the choice of material of the sealing and of the dimensions of the elements of the sealing can also be chosen to provide options for controlling the required forces for locking and sealing the portable base.

It is understood that in the embodiments, wherein the first flange and the second flange define an angle of at least 30° between each other, sealing of the lid unit to the canister unit will happen when the annular skirt is rotated, thereby causing a vertical (downward) movement of the lid unit towards the canister unit. This is turn causes the annular neck portion of the canister unit to engage the abutment section of the second portion of the sealing to incur an upward directed force thereon. At the same time, the lip section of the second portion of the sealing will be forced into engagement with the radially inward facing surface of the annular neck portion of the canister unit and thereby provide a tight seal between the lid unit and the canister unit.

In embodiments, the radially inward facing surface and/or the radially outward facing surface of the annular neck portion is parallel to the central vertical axis. In embodiments, the radially inward facing surface forms an inner annular rim of the annular neck portion. In embodiments, the inner annular rim has an extent in a direction parallel to the central vertical axis of between 5 mm and 30 mm. In one specific implementation, the vertical extent of the inner annular rim is 10 mm. In another specific implementation, the vertical extent of the inner annular rim is 15 mm. In another specific implementation, the vertical extent of the inner annular rim is 20 mm. In yet another specific implementation, the vertical extent of the inner annular rim is 30 mm. Each of these implementations provide a favourable sealing surface for the sealing on the rim portion of the lid unit against the neck portion of the canister unit. In embodiments, an uppermost part of the radially inward facing surface of the neck portion of the canister unit is angularly offset seen relative to the central vertical axis, for example, such that the uppermost part tapers or slopes away from the central vertical axis. This implementation can be useful e.g., for assisting in guidance of the rim portion of the lid unit into correct position on top of the canister unit, in turn guiding the sealing into correct location.

In embodiments, the lip section is configured to seal against the radially inward facing surface of the annular neck portion. In embodiments, the lip section is configured to seal against the radially outward facing surface of the annular neck portion.

In embodiments, the lip section is configured to seal against the radially inward facing surface of the annular neck portion and to seal against a radially outward facing surface of the annular neck portion. In these embodiments, the second portion of the sealing can include two or more flanges whereby at least one flange seals against the radially inward facing surface of the neck portion and at least one other flange seals against the radially outward facing surface of the annular neck portion. Moreover, in these embodiments, the first portion of the sealing can be attached to either the rim portion of the lid unit and/or to another portion of the lid unit than the rim portion. These embodiments can advantageously provide alternative ways of obtaining the sealing effect and/or allow further controlling of the forces required for locking and sealing of the portable base.

In embodiments, an elongated lever arm is attached to the radially inward facing surface of the annular skirt and configured to engagingly cooperate with the at least one projecting jut provided on the radially outward facing surface of the neck portion of the canister unit.

In embodiments, the elongated lever arm comprises a first end portion fixed to the radially inward facing surface of the annular skirt, a connecting portion extending from the first fixed end portion to a second end portion, the connecting portion and the second end portion being spaced from the radially inward facing surface of the annular skirt.

In embodiments, the second end portion of the lever arm is formed as a crosspiece which is substantially transverse to the connecting portion.

In embodiments, the portable base according to the disclosure is configured to be pressurized in the locked configuration. When the portable base is shifted to the locked configuration by partially rotating the annular skirt and engaging of the complementary engagement means, the sealing, being in engagement with the annular neck portion of the canister unit, provides an air- and/or liquid-tight seal between the lid unit and the canister unit. Thus, in the locked (and sealed) configuration, the portable base can be pressurized by an air- (e.g., vacuum-) pump (e.g., incorporated in a system for irrigating the bowels of a user). The pressurization possibility facilitates setting irrigation liquid in the canister unit under a desired operating pressure suitable for irrigating the bowels of a user, by means of an air-pump rather than by using a water-pump for pumping the irrigation liquid out of the portable base and to the anal probe (which would typically work by physically propelling or pushing the liquid). In embodiments, the portable base is configured for a liquid operating pressure of between 6.9-15.9 kPa (approximately 1.0-2.3 psi). In one embodiment, the operating pressure is configured at a maximum of approximately 13.8 kPa (˜2.0 psi). In embodiments, specific implementations of the complementary engagement means can be adapted to help accommodate the desired air-pressurization capacity of the portable base (i.e., adapted to help operate the system with the desired irrigation liquid pressure). Again, such adaptation can be facilitated inter alia by varying e.g., the number of individual threads and/or juts, the tapering/sloping, size/dimensions, and the materials of the complementary means and additionally and/or alternatively by customizing the sealing type and dimensions.

An advantage of the portable base according to the disclosure is that it allows using an air-pump (e.g., vacuum-pump) to pressurize the irrigation liquid in the canister unit. Due to the way the sealing and the means for opening and closing (sealing/unsealing) combine to facilitate (de-) pressurization of the portable base, the invention allows using an air-pump with a system for irrigating the bowels of a user as opposed to using a water (irrigation liquid) pump that physically propels the liquid. Thereby, the use of a water pump for pressurization of the irrigation liquid, and hence the disadvantages involved therewith, is avoided. In a water pump, the irrigation liquid is propelled through the water pump under pressure to irrigate the bowels. Propelling irrigation liquid/water through a water pump incurs a necessary requirement of frequent and thorough cleaning of the water-pump to prevent growth of microorganisms in the pump. With the portable base of the present disclosure, which allows the portable base to be pressurized and reduces the force needed to seal/unseal (close/open) the canister unit, a bowel irrigation system incorporating the disclosed portable base can rely on the use of an air pump and thus avoid sending irrigation liquid through a pump unit. Thereby, the need for frequent cleaning action of the system is in turn avoided. It is thus understood that the disclosure also provides a portable base for a system for irrigating the bowels of a user, in which no physical contact between the irrigation liquid and the pump unit is necessary. This advantageously prevents contamination of the pump unit and further offers a solution wherein no moving parts of the pump unit are subjected to contact with the irrigation liquid or to wear caused by propelling thereof.

In embodiments, the opening of the canister unit has a diameter of between 100-150 mm. In one embodiment, the opening of the canister unit has a diameter of 120 mm. These embodiments are particularly suitable for providing a possibility of thoroughly cleaning the insides of the canister unit, e.g., by allowing a user to insert a hand entirely through the annular opening or alternatively a brush or other cleaning tool.

In embodiments, a radially inward facing surface of the annular neck portion of the canister unit includes at least one set of pairwise mutually spaced projections (meaning that in each set of at least two projections, a first protrusion is spaced from the second protrusion-if more than two projections are applied, each projection is spaced from its neighbouring projection). Each projection has a circumferential length. A radially outward facing surface of the annular rim portion of the lid unit includes at least one set of pairwise mutually spaced indentations that are complementary to the spaced projections (on the annular neck portion). In embodiments, each individual projection in each set of projections is provided with a different circumferential length than its “partner” projection. In embodiments, each individual projection in each set of projections is provided with the same circumferential length as its “partner” projection. In embodiments, each set of projections is provided with a different circumferential length between its “partnering” projections than a neighbouring set of projections. In embodiments, each set of projections is provided with the same circumferential length between its “partnering” projections as a neighbouring set of projections and with specific different circumferential distances to any two neighbouring sets of projections.

In embodiments, a radially inward facing surface of the annular neck portion of the canister unit includes a plurality of spaced projections which are of unequal circumferential lengths and spaced apart by unequal circumferential distances. A radially outward facing surface of the annular rim portion of the lid unit includes a plurality of spaced indentations that are complementary to the spaced projections of the radially inward facing surface of the annular neck portion of the canister unit.

In embodiments, alignment of the complementary spaced indentations of the radially outward facing surface of the annular rim portion of the lid unit with the spaced projections on the annular neck portion of the canister unit provides the aright positioning of the lid unit in relation to the canister unit. When the lid unit is placed on the canister unit over the opening, but without being positioned “aright” on the canister unit, the lid unit in its entirety is easily (with only negligible resistance from friction) rotatable relative to the canister unit. This provides for easy locating of the lid unit onto the canister unit for the user who can simply and easily rotate the entire lid unit (in both clockwise and counterclockwise direction) until it reaches the correct positioning, in which position only the partial rotation of the annular skirt relative to the rim portion will be possible. It is to be understood that such an arrangement of spaced indentations that are complementary to spaced projections ensures that the lid unit can only be placed in the right position on the canister unit, in which position it will be possible to partially rotate the annular skirt relative to the rim portion, when the lid unit is located in precisely the right position in relation to the canister unit. This arrangement therefore prevents any frictional engagement between the (resilient material of the) sealing and the canister unit before the lid unit is correctly (“aright”) located on the canister unit. Apart from providing the functionality described above, the arrangement of the spaced indentations complementary to the spaced projections provides that the user of the portable base is “automatically” directed or guided to place the lid unit correctly on the canister unit, because it will not be possible for the user to rotate the annular skirt and thus move the portable base to the locked configuration unless the lid and canister units are correctly aligned relative to each other. This ensures an intuitive and fail-safe application of the lid unit onto the canister unit to place the portable base in the locked (and sealed) configuration.

In embodiments, an upper portion of a lateral wall of the lid unit includes a through-going passage. In embodiments, the through-going passage provides an opening to a communication port of the lid unit. In embodiments, the through-going passage allows access to a data communication socket of the lid unit. In embodiments, the through-going passage allows access to a power charging socket of the lid unit. In embodiments, the through-going passage provides an opening into a conduit or passageway extending between the surroundings of the portable base and the inside of the lid unit, such as to an internal chamber in the lid unit. The through-going passage in the upper portion of the lateral wall of the lid unit therefore advantageously provides a means for communication between the outside and the inside of the lid unit without having to remove the lid unit from the canister unit and/or without having to otherwise open or gain access to the inside of the lid unit.

In embodiments, the annular skirt includes a screen configured to cover the through-going passage of the lateral wall when the portable base is in the locked configuration. In embodiments the screen is a plate-shaped element provided on the annular skirt and having an axial extent along the central vertical axis such that it covers at least a portion of the upper portion of the lateral wall. In embodiments, the screen is fixed in position to a predetermined location on the radially outward facing surface of the annular skirt. When the annular skirt is partially rotated relative to the annular rim portion of the lid unit, the fixed screen is attached to the annular skirt in a position so that at the end of the rotational movement, the screen is moved to a position relative to the upper portion of the lateral wall of the lid unit wherein it covers the through-going passage. Thereby, the screen advantageously ensures or prevents that the through-going passage can be used for communication with the inside of the lid unit. In one example, the through-going passage can provide access to a power charging socket of the lid unit. The disclosed arrangement of the screen relative to the through-going passage can thereby prevent the possibility of the portable base being ready for an irrigation cycle at the same time where it is attached to an external power source (e.g., mains) for charging. This is advantageous e.g. in that it prevents the portable base being connected to a power source while containing irrigation liquid and thus provides an extra level of security in terms of prevention of contact between liquid (water) and electricity of higher voltages.

In embodiments, the lid unit includes a through-going in-fill passage for filling an irrigation liquid into the volume in the canister unit (i.e., through the lid unit). The through-going in-fill passage is provided generally parallel to the central vertical axis. In embodiments, access to the irrigation liquid passage is provided via a cap. In embodiments, the cap is provided in a top of the lid unit. In embodiments, the cap is a screw cap wherein the cap is screwed onto a threading at the top of the lid unit. It is understood that the cap can be removed/unscrewed to make it possible to fill irrigation liquid into the volume of the canister unit. In embodiments, the cap is attached at a location of the top of the lid unit approximately in the centre of the lid unit. However, the cap can alternatively be located off-centre relative to the centre of the lid unit. In embodiments, the cap comprises an internal sealing (rubber or silicone gasket or gasket-like sealing) for providing an air- and/or liquid-tight seal with the lid unit and in turn the canister unit, to assist in the pressurization of the portable base when it is prepared for an irrigation cycle. The cap facilitates filling (or in some cases emptying) of the canister unit with irrigation liquid without requiring removal or opening (unscrewing/unsealing) of the entire lid unit from the canister unit. Also, the internal sealing of the cap can facilitate (de-) pressurization of the portable base independently of removal of the lid unit from the canister unit which may in turn ease removal of the lid unit from the canister unit.

In embodiments, the lid unit includes at least one internal chamber. The at least one internal chamber is adapted to contain one or more control elements of a system for irrigating the bowels of a user. In embodiments, one or more control elements are held in the internal chamber and/or partially on an outer surface of the lid unit. In embodiments, a top of the lid unit comprises one or more connections and/or interfaces (e.g., spigots or studs) for engagement and correspondence with other elements and/or components of a system for irrigation the bowels of a user. In embodiments, the lid unit includes a passage (e.g., a conduit) for allowing irrigation liquid from the canister unit to exit from the canister, typically by means of a pumping action provided by a pump of the system for irrigating the bowels of a user. In one embodiment, the lid unit includes two separate, internal chambers. In embodiments, the lid unit includes more than two separate, internal chambers. In embodiments, one internal chamber of the lid unit includes or houses a pump and forms part of a system for irrigating the bowels of a user.

In embodiments, the various components of the portable base are made from materials suitable for handling tap water with a temperature of between 5° and 60° Celsius, such as between 30° and 45° Celsius. In embodiments, the canister unit is made from materials suitable for handling temperatures up to 110° Celsius for cleaning purposes. In embodiments, the components of the portable base should be configured to support a system for irrigating the bowels of a user which allows a flow within the levels of approximately 1000 mL/min at 0 bar (0 kPa) and 250 mL/min at 0.1 bar (10 kPa).

The portable base of the disclosure offers the user increased flexibility and increased convenience, particularly, but not exclusively when used in a system for irrigating the bowels of a user. Important factors in those respects include reduction of complexity of the portable base and minimization of requirements for cleaning, drying and other maintenance thereof. Further advantageously, the portable base of the present disclosure can be seen to provide a solution without any electrical connections/engagements between the canister unit and the lid unit, thereby eliminating risks of bad electrical connection and/or other electrical malfunctions. In embodiments, the annular skirt comprises a gripping tab projecting radially outward from the annular skirt. Thus, the gripping tab extends in a direction radially outward in relation to the central vertical axis.

In embodiments, the gripping tab of the annular skirt advantageously extends radially beyond the lateral wall of the lid unit and radially beyond the shoulder portion of the canister unit. A portion of the gripping tab of the annular skirt extending radially beyond the lateral wall and beyond the shoulder portion can be shaped to align with the sidewall of the canister unit. Thereby, access to and movement of the tab is made easy and unobstructed. In embodiments, the gripping tab can have a profile resembling an inverted capital letter “L” with the short leg of the “L” extending radially from the annular skirt and the long leg of the “L” extending axially downwards from the short leg in a direction parallel to the central vertical axis. Alternatively, the long leg of the “L” can extend axially upwards from the short leg in a direction along the central vertical axis. In embodiments, the shape or profile of the gripping tab is designed to both provide and also signal an intuitive handling of the elements of the portable base to the user. Other arrangements and/or shapes/profiles of the gripping tab than the above identified are possible.

In embodiments, the canister unit comprises a finger-hold projecting laterally outward from a sidewall of the canister unit.

In embodiments, the gripping tab of the annular skirt is configured to be aligned with the finger-hold of the canister unit when the portable base is placed in the locked configuration. Thus, the gripping tab on the annular skirt can be configured to generally align with the finger-hold (or second tab) on the sidewall of the canister unit when the portable base is in the locked configuration. Conversely, the gripping tab on the annular skirt and the finger-hold on the canister unit will be configured to not align when the portable base is in the unsealed configuration.

In another aspect, the disclosure relates to a bowel irrigation system including the portable base described herein and further includes the following components: irrigation tubing, at least one anal probe configured to be attached to the irrigation tubing and to the portable base, optionally, a remote control for controlling one or more control elements of the system, optionally, a set of instructions for use, and optionally, a bag for storing the components of the system (between uses). In embodiments, the lid unit of the portable base includes one or more internal chambers as described herein, wherein at least a pump unit and control components or an element adapted to control the irrigation procedure is provided in one or more of the internal chambers. An electrical supply unit for the irrigation system can also be incorporated or can be provided separately from the system itself.

In yet another aspect, the disclosure relates to a method of irrigating the bowels of a user with a bowel irrigation system including the portable base disclosed herein, comprising the steps of:
(a) providing the canister unit and the lid unit of the portable base;
(b) providing the additional components of a bowel irrigation system;
(c) providing a volume of irrigation liquid in the canister unit;
(d) placing the lid unit on the neck portion of the canister unit to close the annular opening of the canister unit;
(e) rotating the annular skirt on the rim portion of the lid unit such that the at least one thread of the annular skirt and the at least one jut of the canister unit engage with each other to shift the portable base to a locked configuration; and
(f) irrigating the bowels of the user.

It is understood that the step of rotating the annular skirt does not involve a full rotation of the annular skirt, but merely involves causing the annular skirt to be turned slightly, or at least partially, around the central vertical axis of the portable base.

In yet another aspect, the disclosure relates to a method of sealing a canister unit of a bowel irrigation system including the portable base disclosed herein, comprising the steps of:
(a) providing the canister unit and the lid unit of the portable base;
(b) providing the additional components of a bowel irrigation system;
(c) providing a volume of irrigation liquid in the canister unit;
(d) placing the lid unit on the neck portion of the canister unit to close the annular opening of the canister unit; and
(e) rotating the annular skirt on the rim portion of the lid unit such that the at least one thread of the annular skirt and the at least one jut of the canister unit engage with each other to shift the portable base to a locked configuration.

As above, it is understood that the step of rotating the annular skirt does not involve a full rotation of the annular skirt, but merely involves causing the annular skirt to be turned slightly, or at least partially, around the central vertical axis of the portable base.

The method according to this aspect is advantageous in that using a portable base as disclosed above provides the effects and benefits to the user described in detail for the portable base device, including (but not limited to) ease of sealing and opening the canister unit using significantly reduced physical force, and further providing significantly easier access for cleaning of the canister unit.

### DETAILED DESCRIPTION OF THE DRAWINGS

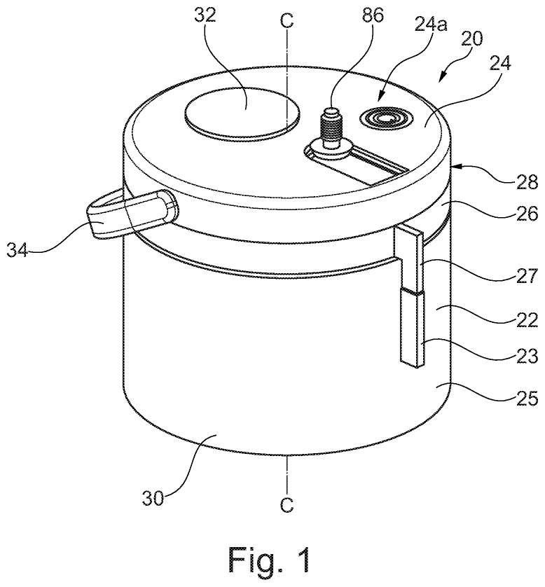

FIG. 1 is concerned with a first aspect of the present invention and is a perspective view illustrating one embodiment of a portable base 20 for a system for irrigating the bowels of a user. The portable base includes a canister unit 22, which is self-supporting and a lid unit 24. In the perspective view of FIG. 1, the portable base 20 is shown in a locked (sealed) configuration with the lid unit 24 placed “on top of” and closing the canister unit 22. The canister unit 22 is adapted to hold a volume of irrigation liquid (not shown) inside the canister. It is to be understood that the lid unit 24 is removably connected to the canister unit 22. The lid unit 24 includes an annular skirt 26. The portable base 20 is illustrated in the locked configuration in which the lid unit 24 is adapted to be released and subsequently removed from the canister unit 22 by partially rotating the annular skirt 26. The annular skirt 26 is partially rotatable independently of other portions or parts of the lid unit 24 when the lid unit 24 is positioned aright on the canister unit 22. When the lid unit 24 is free from engagement with the canister unit 22 (i.e. “lifted off”), the annular skirt 26 is secured in relation to other portions or parts of the lid unit and cannot be rotated or moved relative thereto. A gripping tab 27 extends out from the annular skirt 26 and is illustrated to substantially align with a finger-hold 23 of the canister unit 22. Particularly, the gripping tab 27 and the finger-hold 23 of the canister unit are configured to align with each other in the locked configuration of the portable base 20. In FIG. 1, the gripping tab 27 is shown to have an exemplary profile basically resembling an inverted capital letter “L” with the short leg of the “L” extending radially from the annular skirt 26 and the long leg of the “L” extending axially downwards from the short leg in a direction parallel to the central vertical axis. The dimensions of the “legs” of the “L” are illustrated exaggeratedly in FIG. 1. By grasping and moving the gripping tab 27 of skirt 26 and holding for example the fingers of the other hand on the finger-hold 23 of the canister unit 22, the annular skirt 26 can be partially rotated in a direction around the central vertical axis C-C and be released from engagement with the canister unit 22 as complementary engagement means (thread(s)/jut(s), not illustrated in FIG. 1) provided on the annular skirt 26 and on the annular neck portion of the canister unit 22 release from each other. After such rotational action of the annular skirt 26, and the ensuing release of at least one thread of the annular skirt 26 from at least one jut 62 of the canister unit 22, the lid unit 24 can be removed from the canister unit 22 by lifting it off therefrom. In the embodiment shown in FIG. 1, the lid unit 22 includes a handle 34 attached to the lid unit which can be used when lifting off the lid unit 24 from the canister unit 22 and for moving of the portable base when it is in the locked configuration.

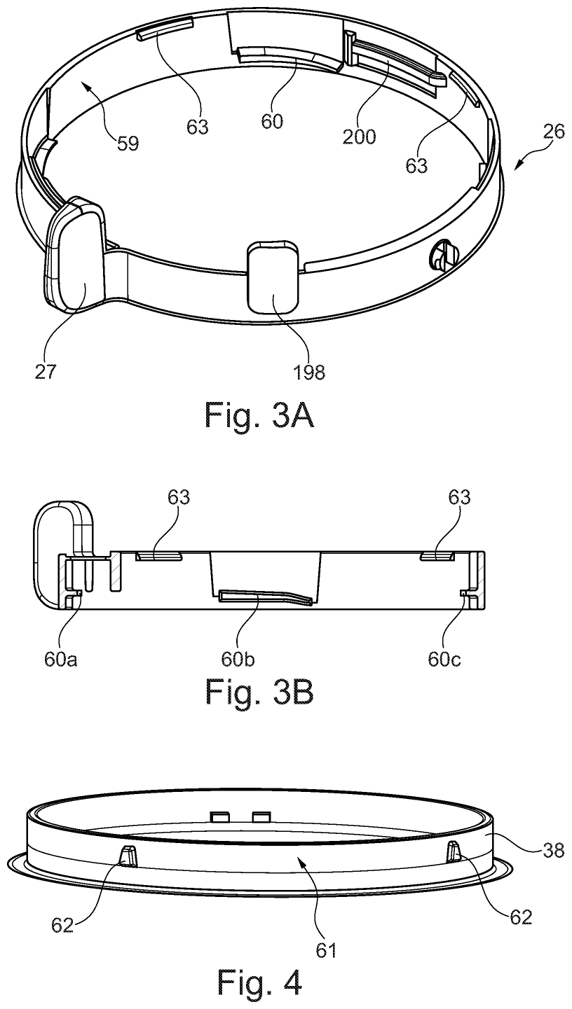

The lid unit 24 can be configured to hold one or more control elements or control components of a bowel irrigation system. The lid unit 24 includes a lateral wall 28. In the illustrated embodiment, the lateral wall 28 forms an annular sidewall of the lid unit 24. In the illustrated embodiment, the finger-hold 23 of the canister unit extends laterally out from a sidewall 30 of the of the canister unit 22. Further, the lid unit 24 is shown to include a cap 32 which can be opened to supply an irrigation liquid through an opening in the lid unit 24 (opening not visible in FIG. 1 as it is covered by the cap 32) and into the volume of the canister unit 22. The cap 32 is shown to be provided on top surface 24a of the lid unit 24 and is removable therefrom, e.g., by unscrewing of the cap 32 from a threaded connection in the lid unit. In FIG. 1, the sidewall 30 of the canister unit 22 is illustrated to be substantially straight and parallel to the central vertical axis C-C of the portable base 20. In FIG. 1, the handle 34 is illustrated to be attached to the upper portion of the lateral wall 28 of the lid unit 24, however the handle 34 can also be attached to the annular skirt 26 (cf. at least FIG. 3A). The opening and/or closing actions of the portable base 20 by partially rotating the annular skirt 26 can conveniently be carried out by pushing the gripping tab 27 in one direction with the fingers of one hand and holding on to the finger-hold 23 of the canister unit 22 with the fingers of the other hand.

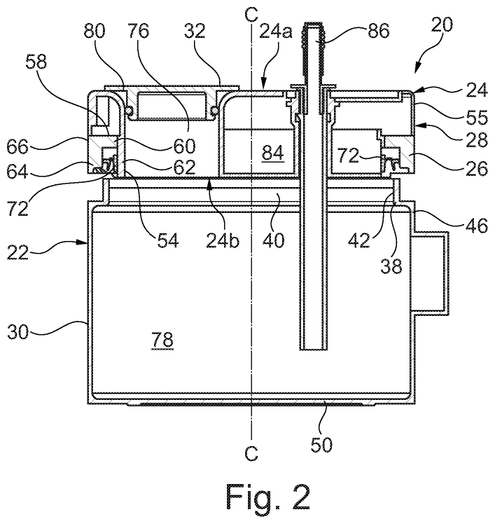

With reference to the embodiments illustrated in FIGS. 1 and 2, the lid unit 24 includes a through-going passage 76 for filling irrigation liquid into the volume 78 in the canister unit 22. The through-going passage 76 is provided generally parallel to the central vertical axis C-C. Access to the irrigation liquid passage 76 is provided via cap 32. The cap 32 is provided in the top 24a of the lid unit 24 and can be attached to the top of the lid unit 24 e.g. by a threaded connection of by a press-fit connection. As shown FIG. 2, the cap 32 can include an internal sealing 80 for assisting in providing the air- and liquid-tight seal between the lid unit 24 and the canister unit 22 in use of the portable base 22. The cap 32 facilitates filling (or emptying) of the canister unit 22 with irrigation liquid without requiring entire removal of the lid unit 24 from the canister unit 22. In embodiments as illustrated in FIG. 2, the lid unit 24 includes an internal chamber 84 advantageously adapted to contain one or more control elements (not shown) of a system for irrigating the bowels of a user. With reference to both FIGS. 1 and 2, on the top 24a of the lid unit 24 a connection 86 for engagement with tubing of a system for irrigation of the bowels of a user is provided. The connection 86 also forms a passage for allowing the irrigation liquid from the canister unit 22 to exit by means of a pumping action provided by a pump of the system for irrigating the bowels of a user (which could conveniently be in the internal chamber 84).

FIG. 2 is a cross-sectional view of one embodiment of the portable base 22 in an unlocked and unsealed/open configuration, wherein the lid unit 24 is free from engagement with the canister unit 22. The canister unit 22 includes an annular neck portion 38 which in the illustrated view circumscribes an opening 40 around the central vertical axis C-C of the portable base 20. The annular neck portion 38 includes a vertical flange 42 extending parallel to the central vertical axis C-C. The annular neck portion 38 transitions into a shoulder portion 46 of the canister unit 22. The transition between the annular neck portion 38 and the shoulder portion 46 is located at a lower part of the annular neck portion 38. The shoulder portion 46 of the canister unit 22 transitions into sidewall 30 of the canister unit 22. The transition between the shoulder portion 46 and the sidewall 30 of the canister unit 22 is at a lower part of the shoulder portion 46. In the embodiment of FIG. 2, the annular neck portion 38 is offset (or extends) radially inwardly from the sidewall 30 of the canister unit 22 at the shoulder portion 46 and is therefore located radially closer to the central vertical axis C-C than is the sidewall 30 of the canister unit 22.

The annular neck portion 38 of the canister unit 22 generally circumscribes the opening 40. The annular neck portion can be seen to delimit the extent of (and thus a size) the opening 40 of the canister unit 22.

In FIG. 2, the lid unit 24 is shown “on top” of the canister unit 22 and ready for closing of the opening 40. The lid unit 24 has an axial extent along the central vertical axis C-C between a top 24a and a bottom 24b of the lid unit 24. The lid unit 24 includes an annular rim portion 54. The lid unit 24 further includes the annular skirt 26. The annular skirt 26 is at least partially rotatable relative to the annular rim portion 54. The annular rim portion 54 can be understood to form part of the lateral wall 28 of the lid unit 24. In FIG. 2, the annular rim portion 54 forms a lower portion of the lateral wall 28 (seen in relation to positions on the central vertical axis C-C). In the illustrated embodiment, the annular rim portion 54 is parallel to the central vertical axis C-C and is offset radially inward in relation to an upper portion 55 of the lateral wall 28 seen in relation to the central vertical axis C-C. In the embodiment of FIG. 2, the lateral wall 28 of the lid unit 24 also defines a shoulder 58 at a transition between the annular rim portion 54 and the upper portion 55 of the lateral wall 28.

FIG. 2 further illustrates a location of a sealing 72 provided at the annular rim portion 54 to seal between the canister unit 22 and the lid unit 24 in the locked configuration of the portable base 20. The sealing 72 is configured to be moved by a force exerted by the annular neck portion 38 of the canister unit 24 when the user rotates the annular skirt 26 relative to the rim portion 38. In FIG. 2, the sealing 72 is shown to be attached to the annular rim portion 54 of the lid unit 24. The sealing 72 is configured to be brought into an initial abutment with the annular neck portion 38 of the canister unit 22 when the lid unit 24 is positioned aright on the annular neck portion 38. A seal (sealing effect) is effectuated when the sealing 72 is urged or pressed into engagement with the annular neck portion 38 by rotation of the annular skirt 26 to cause the juts 62 and threads 60 (FIGS. 3A, 3B, 4) to engage with each other and shift the portable base 20 to the locked configuration. The engagement of the juts 62 and the threads 60 with each other resulting from the user's rotational movement of the annular skirt 26 provides an axially downward movement of the entire lid unit 24 in relation to the canister unit 22. This in turn causes the sealing 72 to be pressed into leak-tight engagement with the annular neck portion 38 and creates a seal (sealing effect) between the lid unit 24 and the canister unit 22.

In the schematic perspective view of FIG. 3A, a radially inward facing surface 59 of the annular skirt 26 of the lid unit 24 includes at least one thread 60 configured to engage with complementary engagement means on the annular neck portion 38 of the canister unit 22 (see FIG. 4). FIG. 3B is a schematic cross-sectional view of the annular skirt 26 of FIG. 3A.

FIG. 4 is a schematic perspective view illustrating the annular neck portion 38 at a top end of the canister unit. FIG. 4 illustrates complementary engagement means to the thread 60 of FIG. 3A in the form of at least one, in this case two, projecting juts 62 provided on a radially outward facing surface 61 of the annular neck portion 38 of the canister unit 22.

With general reference to the embodiments illustrated in FIGS. 3A, 3B and 4, the radially inward facing surface 59 of the annular skirt 26 includes four threads of which three 60a, 60b, 60c are visible. The radially inward facing surface 59 is provided by (or on) the peripheral wall of the annular skirt 26. The threads 60a, 60b, 60c extend in a direction parallel to a plane which is generally perpendicular to, and radially inward towards, the central vertical axis of the portable base (FIG. 1). The illustrated threads 60 are formed as flanges or flange-like elements having an extent along the radially inward facing surface 59 of the peripheral wall. Looking at thread 60b in FIG. 3B, it is possible to see how the flange of the thread varies in dimension and slope. The right-hand end portion (when viewed in FIG. 3B) of the thread 60b is shown to have a different slope or inclination than other portions of the thread 60b. The sloped/inclined or tapered threads 60 engage with the juts 62 such that by the rotational movement of the annular skirt 26 relative to the annular rim portion 54, the lid unit 24 is moved in an axial direction parallel to the central vertical axis.

The radially outward facing surface 61 of the annular neck portion 38 of the canister unit 22 in FIG. 4 includes four complementary projecting juts 62 of which two are visible in FIG. 4. The four threads 60 on the radially inward facing surface 59 of the annular skirt are configured to cooperate with the four complementary projecting juts 62 on the radially outward facing surface 61 of the annular neck portion 38 of the canister unit 22. The threads 60 and the projecting juts 62 are configured to go into engagement with each other to attach the lid unit 24 to the canister unit 22 in the locked configuration of the portable base 20. The resulting downward movement of the lid unit 24 causes the annular neck portion 38 of the canister unit 22 to engage with and move the sealing 72. As a simultaneously occurring effect, the sealing 72 is pressed (squeezed) against the annular neck portion 38.

FIGS. 3A and 3B further illustrate embodiments, in which the radially inward facing surface 59 of the annular skirt 26 includes four radially inward extending support flanges 63 of which two are visible in FIGS. 3A and 3B. The support flange(s) 63 are configured to be located and rest on a ledge of the lid unit 24 when the annular skirt 26 is attached to the remainder of the lid unit 24. The engagement between the support flanges 63 of the annular skirt 26 and the ledge of the lid unit 24 provides for the support flange to slide on the ledge and help provide for the at least partial rotation of the annular skirt 26 relative to the annular rim portion 54 of the lid unit 24.

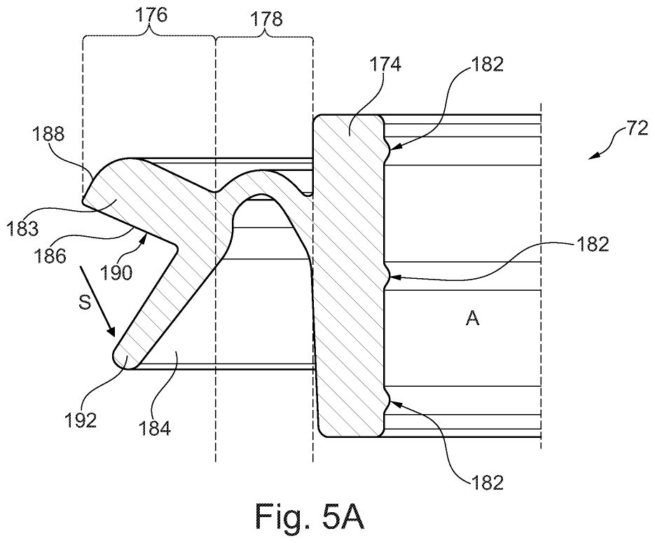

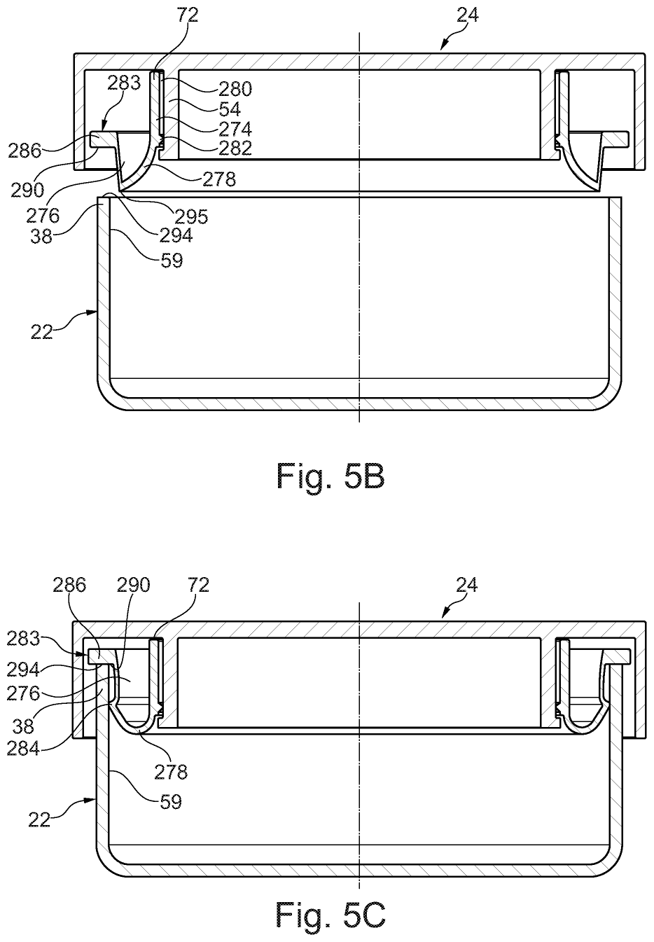

FIG. 5A illustrates one embodiment in which the sealing 72 has a first portion 174 configured to be fixed to the annular rim portion 54 of the lid unit 24 and a second portion 176 configured to engage with the annular neck portion 38 of the canister unit 22. FIG. 5A shows a schematic sectional perspective view of a portion or section of the sealing 72 provided as an annular ring to be attached around the annular rim portion 54 of the lid unit 24. The first portion 174 of the sealing 72 includes a radial inner surface 180 including three radially inward extending protrusions 182 marked by arrows A which form “packing-” or sealing surfaces of the sealing 72 against the annular rim portion 54. The sealing 72 has a connecting portion 178 extending between the first portion 174 and the second portion 176. In the embodiment of FIG. 5A, a material thickness of the connecting portion 178 is less than a material thickness of the first portion 174. The connection portion 178 is shown to have an arched profile between the first and second portions.

The second portion 176 of the sealing 72 includes an abutment section 183 configured for engagement with a top surface of the annular neck portion 38 of the canister unit 22. The second portion 176 also has a lip section 184 configured to seal against the radially inward facing surface 59 of the annular neck portion 38 of the canister unit 22. In FIG. 5A, the abutment section 183 includes a flange 186 extending from the connecting portion 178 of the sealing 72. The flange 186 has a partially rounded-off end portion 188. A downwardly facing surface 190 of the flange 186 is generally flat or level and adapted to engage with the top surface (not illustrated in FIG. 5A) of the annular neck portion 38. The lip section 184 includes another flange 192 extending from the connecting portion 178. In this configuration of the sealing 72, the abutment section 183 of the second portion 176 is subjected to and picks up the movement-incurring force and the lip section 184 provides the sealing effect. The first flange 186 of the abutment section 183 and the second flange 192 of the lip section 184 define an angle between each other. In the embodiments of FIG. 5A, the angle is approximately 85°.

FIGS. 5B-5C illustrate another embodiment in which the sealing 72 has a first portion 274 configured to be fixed to the annular rim portion 54 of the lid unit 24 and a second portion 276 configured to engage with the annular neck portion 38 of the canister unit 22. FIGS. 5B-5C show a schematic sectional view of the portable base 20 with sealing 72 provided as an annular ring to be attached around the annular rim portion 54 of the lid unit 24. The first portion 274 of the sealing 72 includes a radial inner surface 280 including one or more radially inward extending protrusions 282 (one shown in FIGS. 5B-5C) which form a “packing-” or sealing surface of the sealing 72 against the annular rim portion 54. The sealing 72 further has a connecting portion 278 extending between the first portion 274 and the second portion 276. In the embodiment of FIGS. 5B-5C, a thickness of the connecting portion 278 is slightly less than a thickness of the first portion 274. The connection portion 278 is shown with an arched profile between the first and second portions. FIG. 5B shows the portable base in the unlocked configuration of the portable base 20, i.e., before sealing the lid unit 24 to the canister unit 22. The canister unit 22 and the lid unit 24 of the embodiment of FIGS. 5B-5C are configured with at least partly different shapes and features than the embodiments of FIGS. 1 and 2.

The second portion 276 of the sealing 72 of FIGS. 5A-5C includes an abutment section 283 configured for engagement with a top surface 294 of the annular neck portion 38 of the canister unit 22. An apex 295 of a transition between the second portion 276 and the connecting portion 278 forms a lip section 284 configured to seal against a radially inward facing surface 59 of the annular neck portion 38 of the canister unit 22. In FIGS. 5C, the flange 286 of abutment section 283 extends at an angle relative to a remainder of the second portion 276. A downwardly facing surface 290 of the flange 286 is generally flat or level and adapted to engage with the top surface 294 of the annular neck portion 38. In this configuration of the sealing 72, the abutment section 283 of the second portion 276 is subject to and picks up the movement-incurring force and the lip section 284 provides the sealing effect. FIG. 5C shows the portable base 20 in the locked configuration, i.e., after the annular skirt has been partially rotated to seal the lid unit 24 onto the canister unit 22. In FIGS. 5B-5C the connecting portion 278 is illustrated to be connected to the first portion 274 at or near a lower end of the first portion 274, at which lower end also the protrusion 282 “packing” against the annular rim portion 54 is provided. This configuration provides a beneficial column strength to the first portion 274, which in turn increases the sealing strength or effect at the apex 295 of the lip section 284 in the locked configuration of the portable base 20.

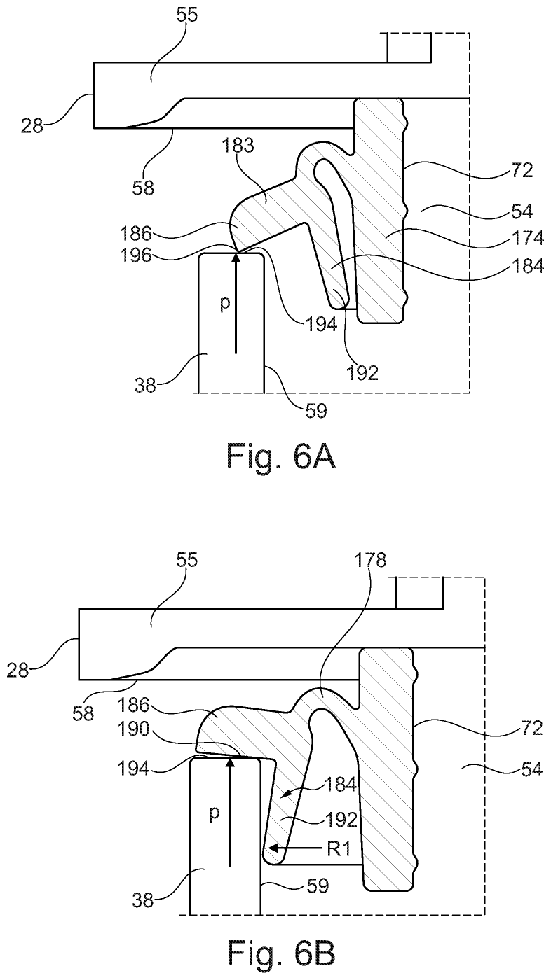

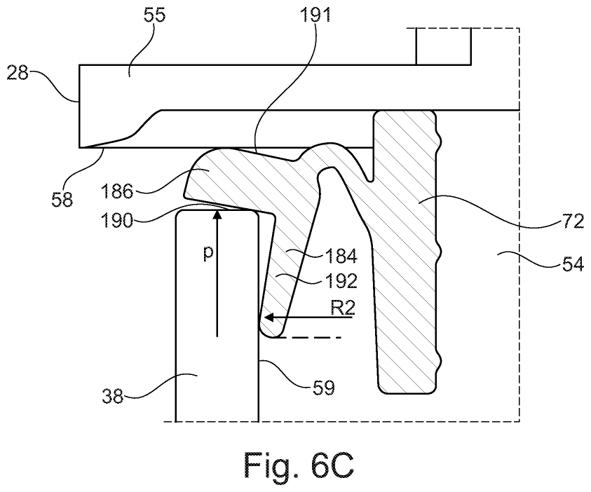

FIGS. 6A, 6B and 6C are schematic sketches illustrating sectional views of the sealing arrangement of the portable base 20, and particularly the sealing 72, in three different positions according to one embodiment thereof. FIGS. 6A, 6B and 6C are merely intended to illustrate how the force incurred by the user's rotational movement of the annular skirt 26 causes the sealing 72 to move and seal against the annular neck portion 38 of the canister unit 22. FIG. 6A shows the sealing 72 in a rest position corresponding to the “aright” position of the lid unit 24 on the canister unit 22. In the rest position of FIG. 6A, the flange 186 of the abutment section 183 of the second portion of the sealing 72 contacts a top surface 194 of the annular neck portion 38 at an edge 196 of the flange 186. The flange 192 of the lip section 184 is not engaged with the annular neck portion 38 of the canister unit 22. The first portion 174 of the sealing 72 is attached to the annular rim portion 54 of the lid unit 24. Shoulder 58 between the annular rim portion 54 and the upper portion 55 of a lateral wall 28 of the lid unit is also visible. Arrow P indicates the direction of the force incurred on the abutment section 183 resulting from the user's rotational movement of the annular skirt 26 (not shown in FIG. 6A-6C) around the annular rim portion 54.

In the view of FIG. 6B, the sealing 72 is shown in an intermediate position in which the user has rotated the annular skirt 26 somewhat around the annular rim portion 54, but the portable base 20 has not yet reached the locked configuration. It can be seen how the rotational movement has caused the lid unit to move closer to the canister unit, i.e. observing how the shoulder 58 is closer to the annular neck portion 38 in FIG. 6B than in FIG. 6A. The top surface 194 of the annular neck portion 38 engages with the downwardly facing surface 190 of the flange 186. Due to this arrangement of the sealing 72, the upward directed force exerted on the sealing 72 by the annular neck portion 38 causes the connection portion 178 of the sealing 72 to deform and the flange 192 of the lip section 184 to engage with and begin to seal against the radially inward facing surface 59 of the annular neck portion 38 by a force indicated in direction and relative magnitude by arrow R1. In the view of FIG. 6C, the sealing 72 is shown in the final sealing position, corresponding to the locked configuration of the portable base 20, in which the user has finished the partial rotation of the annular skirt 26 around the annular rim portion 54. In FIG. 6C, it can be seen how the rotational movement has caused the lid unit to move even closer to the canister unit, i.e. observing how the shoulder 58 is closer to the annular neck portion 38 in FIG. 6C than in FIG. 6B and even engages with an upwardly facing surface 191 of the flange 186. The continued upward direction of force exerted on the sealing 72 by the annular neck portion 38 has now caused the connection portion 178 of the sealing 72 to deform even further (and can even be seen to also abut/engage the shoulder 58). The flange 192 of the lip section 184 is now in firm engagement with the radially inward facing surface 59 of the annular neck portion 38 and has created a strong seal there against. The increased force and thus strength of the sealing effect (compared to the intermediate position of the sealing 72 in FIG. 6B) is illustrated by relative greater arrow R2 compared to arrow R1 in FIG. 6B. FIG. 6C further illustrates the increased strength of the seal in that flange 192 of the lip section 184 is shown to be deformed having a slight arched profile. Again, it should be understood that when the sealing 72 is urged or pressed into engagement with the radially inward facing surface 59 of the annular neck portion 38 by rotation of the annular skirt 26 to make the complementary juts 62 and threads 60 come into engagement with each other, the sealing effect is created and the portable base 22 is shifted to the locked (and sealed) configuration. The rotation of the annular skirt 26 and the resulting downward, vertical movement of the lid unit 24 leads to a pressure being applied on the material of the sealing 72 sufficient to create an air- and liquid-tight seal between the lid unit 24 and the canister unit 22.

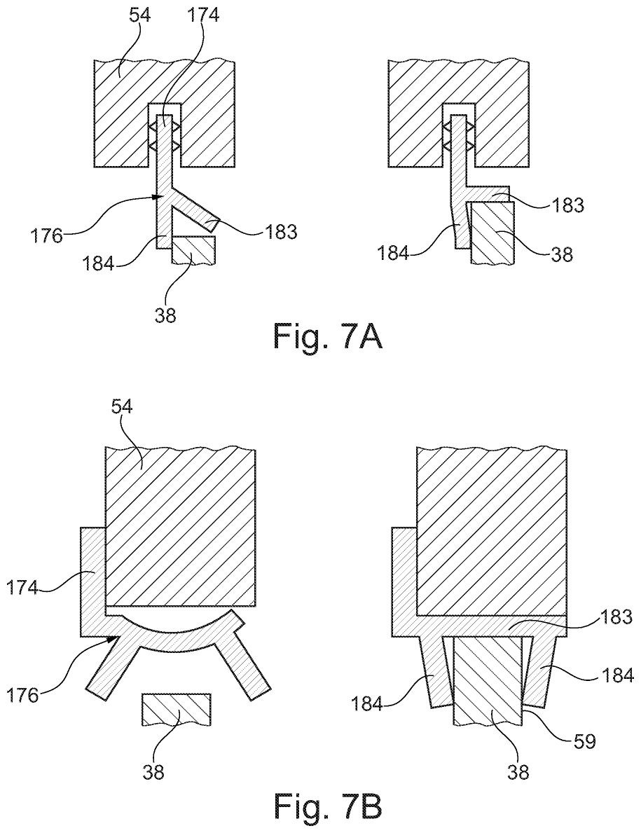

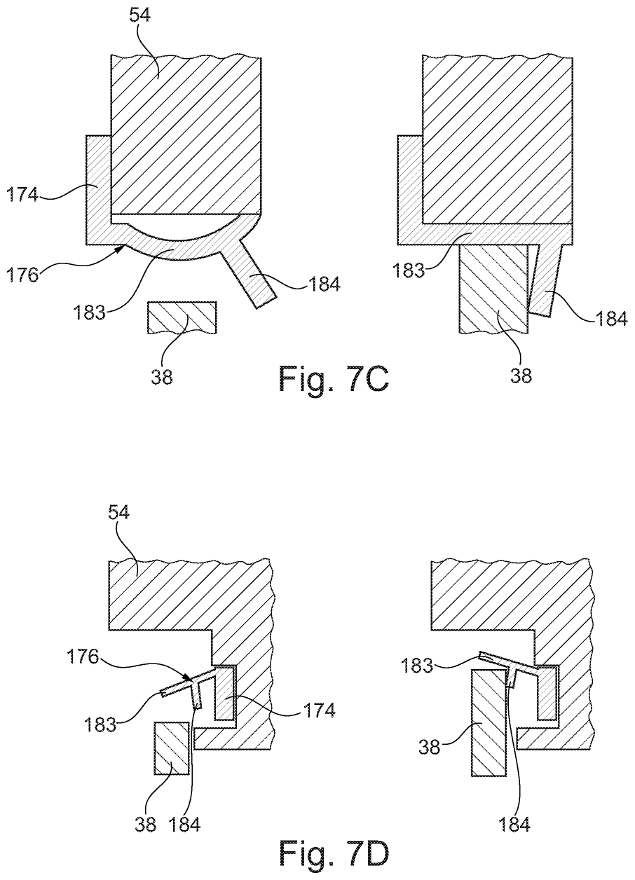

FIGS. 7A-7D are schematic sketches illustrating in cross-section four alternative configurations or embodiments of the sealing 72. The left-hand side of each of the sketches shows the sealing 72 in a position in which no force is exerted on the sealing. The right-hand side show each of the sealing embodiments in a deformed position wherein a force is exerted on the sealing. In each case the sealing 72 has first portion 174 attached to the annular rim portion 54 of the lid unit 24. The sealing 72 further has a second portion 176 configured to engage with the annular neck portion 38 of the canister unit 22. In each embodiment, the second portion 176 includes an abutment section 183 for abutting the annular neck portion 38 and at least one lip section 184 (in FIG. 7B the second portion 176 has two lip sections 184, configured to engage the radially inward facing surface 59 of the annular neck portion 38 and the radially outward facing surface of the annular neck portion 38, respectively) for sealing against the annular neck portion 38 of the canister unit 22.

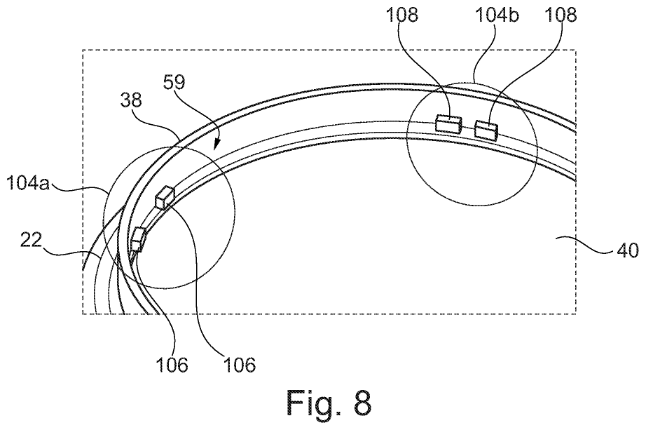

FIG. 8 is a schematic, perspective sectional view of one embodiment showing a section of the annular neck portion 38 provided around the opening 40 of the canister unit 22. The annular neck portion 38 includes radially inward facing surface 59 on which two sets 104a, 104b of pairwise mutually spaced projections 106, 108 can be seen. In FIG. 8, a first set of projections 104a has a circumferential length between its “partnering” projections 106 different from a neighbouring second set 104b of projections 108. In FIG. 8, the first set 104a of projections 106 are provided with a relatively longer distance between its projections 106 than the second set 104b of projections 108, i.e. there is a shorter distance between the two projections marked 108 than between the two projections marked 106. It is understood that in embodiments more than two sets of projections can be applied and that also only one set of pairwise mutually spaced projections can be applied on the annular neck portion 38 of the canister unit 22.

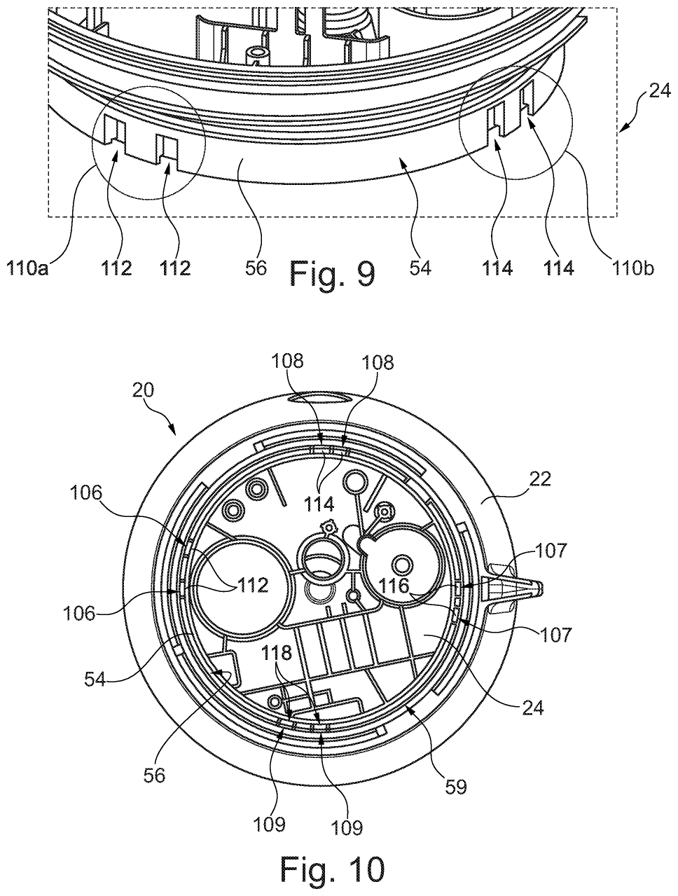

FIG. 9 is a schematic, perspective sectional view of one embodiment showing a section of the lid unit 24 and particularly the annular rim portion 54. The annular rim portion 54 includes radially outward facing surface 56 on which two sets 110a, 110b of pairwise mutually spaced indentations 112, 114 are provided. In FIG. 9, a first set of indentations 110a is provided with a different circumferential length between its “partnering” indentations 112 than a neighbouring second set 110b of indentations 114. In FIG. 9, the first set 110a of indentations 112 are provided with a relatively longer distance between the indentations 112 than the second set 110b of indentations 114, i.e. there is a longer distance between the two indentations marked 112 than between the two indentations marked 114. It is understood that in embodiments more than two sets of indentations can be applied to the annular rim portion 54 of the lid unit 24 and that also only one set of pairwise mutually spaced indentations can be applied on the annular rim portion 54.

It will be understood that the sets 110a, 110b of pairwise mutually spaced indentations 112, 114 provided on the radially outward facing surface 56 of the annular rim portion 54 of the lid unit are complementary to the sets 104a, 104b of pairwise mutually spaced projections 106, 108 of the radially inward facing surface 59 of the annular neck portion 38 of the canister unit 22. Due to the disclosed arrangement of the projections 106, 108 and indentations 112, 114 the lid unit 24 can only fit correctly onto the canister unit 22 in the “aright” position wherein the complementary projections and indentations exactly match each other. Thus, the lid unit 24 can only be brought into aright position on the canister unit 22 when the complementary means are correctly aligned. This includes matching the sets of projections with the sets of indentations having the same distance between them. Thereby, controlling exactly when (in what position) the annular skirt 26 can be rotated relative to the annular rim portion 54 of the lid unit, i.e. only in the correctly aligned configuration, is achieved. The projections 106, 108 and indentations 112, 114 can be considered keys and slots, which need to fit together in the correct match to make the lid unit 24 come into correct (aright) position on the canister unit 22. As long as the lid unit 24 is placed on the canister unit over the opening without being positioned “aright” on the canister unit, i.e. with the keys and slots fitting exactly into each other, the lid unit 24 can be easily rotated in its entirety relative to the canister unit (with no or only very little resistance from friction because the sealing 72 is not or only in negligible contact with the canister unit 22).

FIG. 10 is a top view of one embodiment of the portable base 20 with the lid unit 24 positioned on top of the canister unit 22, but with top 24a (cf. FIG. 1) of the lid unit 24 removed for illustration purposes. Four sets of pairwise mutually spaced indentations 112, 114, 116, 118 are provided on the radially outward facing surface 56 of the annular rim portion 54 of the lid unit. These are complementary to four sets of pairwise mutually spaced projections 106, 107, 108, 109 of the radially inward facing surface 59 of the annular neck portion 38 of the canister unit 22.

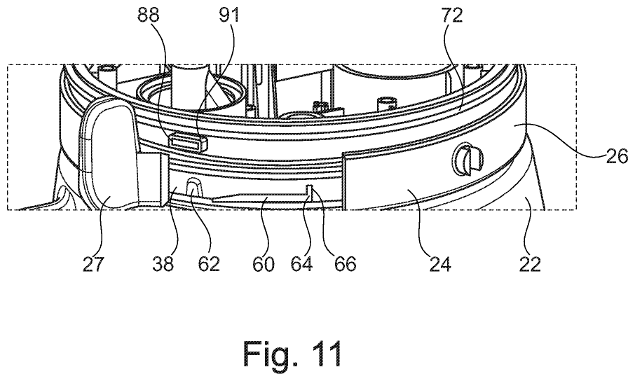

FIG. 11 is a schematic perspective view of a portion of the lid unit 24 in position on top of the canister unit 22 in one embodiment of the portable base 20. For illustration purposes, various elements of the lid unit 24 have been removed in the view of FIG. 11. These include: a portion of the peripheral wall of the annular skirt 26; the upper portion 55 of the lateral wall 28, and the top 24a of the lid unit 24. This reveals through-going passage 88. In the illustrated embodiment of FIG. 11, the through-going passage 88 provides an opening to a communication port 91 of the lid unit 24, such as a data communication socket and/or a combined data communication and power charging socket of the lid unit 24. It is understood that the opening of the through-going passage 88 is an opening in the upper portion 55 of the lateral wall 28, however this is not shown in the view to better illustrate the communication port 91. Also visible in FIG. 11 is the sealing 72 in the form of a ring extending around the annular rim portion 54 of the lid unit 24 and a gripping tab 27 of the annular skirt 26. Also visible in FIG. 11 is a jut 62 provided on the radially outward facing surface 56 of the annular neck portion 38 of the canister unit 22. For illustration purposes, a thread 60, which is a complementary engagement means to the jut 62, is also illustrated. However, it is to be understood that the thread 60 is in practice provided on the radially inward facing surface 59 of the portion of the peripheral wall of the annular skirt 26 (which has been removed in the view of FIG. 11 to better illustrate the position of the thread 60 for engagement with the jut 62). In the embodiment of FIG. 11, an end portion 64 of the thread 60 is shown to include a generally vertical stop 66.

Referring again to the embodiment of the annular skirt 26 illustrated in the view of FIG. 3A, the annular skirt 26 can be seen to include a screen 198. The screen 198 is configured to cover the through-going passage 88 in the upper portion 55 of the lateral wall 28 of the lid unit 24 when the portable base 20 is in the locked configuration. Considering both the views of FIG. 3A and FIG. 11, it will be understood that when the annular skirt 26 is partially rotated relative to the annular rim portion 54 of the lid unit 24, the screen 198 is moved to a position over the upper portion 55 of the lateral wall 28 of the lid unit 24 in which the screen 198 covers the through-going passage 88 at the end of the partial rotational movement. Thereby, the screen 198 prevents that the through-going passage 88 can be used for communication with the inside of the lid unit 24 (e.g. prevents power charging of a rechargeable battery which can suitably be provided in an internal chamber 84, FIG. 2) when the portable base 20 is in the locked configuration.

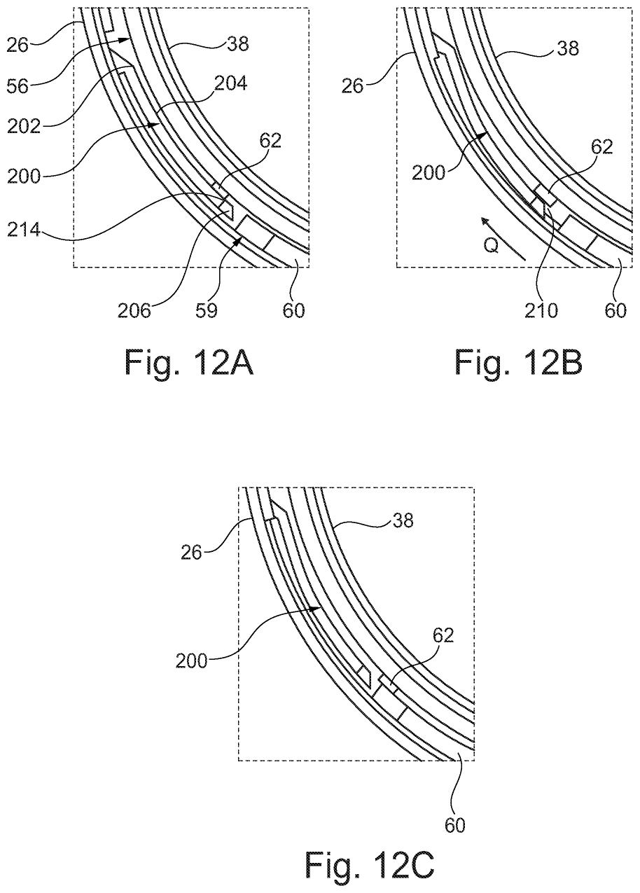

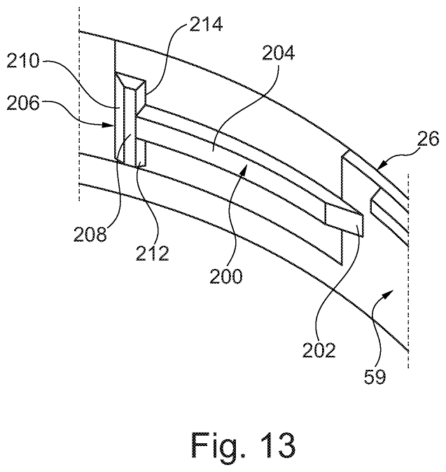

FIGS. 12A, 12B and 12C are schematic top views illustrating a section of the annular neck portion 38 of the canister unit 22 with the annular skirt 26 of the lid unit 24 radially outward thereof. In each of the views of FIGS. 12A, 12b and 12C, a jut 62 provided on the radially outward facing surface 56 (FIG. 12A) of the annular neck portion 38 and a portion of a thread 60 provided in the radially inward facing surface 59 (FIG. 12A) of the annular skirt 26 are also visible. In the embodiments illustrated in FIGS. 12A-12C, an elongated lever arm 200 is shown to be attached to the radially inward facing surface 59 of the annular skirt 26. The elongated lever arm 200 is configured to engage and cooperate with the projecting jut 62 on the radially outward facing surface 56 of the annular neck portion 38 of the canister unit 22. In the embodiments illustrated in FIGS. 12A-12C, the elongated lever arm 200 has a first end portion 202 fixed to the radially inward facing surface 59 of the annular skirt 26. The elongated lever arm 200 also has a connecting portion 204 extending from the first fixed end portion 202 to a second end portion 206. As can be seen particularly in the views of FIGS. 12A and 12C, the connecting portion 204 and the second end portion 206 are spaced from the radially inward facing surface 59 of the annular skirt 26. In the embodiments illustrated in FIGS. 12A-12C, and further with reference to FIGS. 3A and particularly FIG. 13, which is an enlarged view of a portion of the annular skirt 26 of FIG. 3A, the second end portion 206 of the lever arm 200 includes a crosspiece 208 provided transverse to the connecting portion 204 at one end thereof. In the embodiment of FIG. 13, externally facing surfaces of the crosspiece 208 include a variety of differently shaped and/or tapered or sloped/inclined edges. A first surface 210 is illustrated to be provided with a first tapering. A second surface 212 is illustrated to be provided with a second tapering. A third surface 214 is illustrated to not have a tapering, but instead a straight edge which can be understood to be an edge/surface extending substantially in a radial direction of the portable base 20. In embodiments, the straight edge third surface 214 of the crosspiece is configured to cooperate with a stop on the lid unit 24 to entirely prevent rotation of the annular skirt 26 relative to the annular rim portion 54 of the lid unit 24 when the lid unit 24 is not positioned on top of the canister unit 22. In embodiments as illustrated in FIG. 13, the elongated lever arm 200 can generally be understood to form a hammer shape with the hammerhead (crosspiece 208) and the shaft of the hammer (connection portion 204) extending freely and with a base (first end portion 202) being fixed to the annular skirt 26. The hammerhead and the shaft of the hammer are spaced from the annular skirt 26 with air therebetween, at least in an unbiased position of the elongated lever arm 200.

Referring again now particularly to FIGS. 12A-12C, FIG. 12A illustrates the arrangement of the elongated lever arm 200 in a first unbiased position in which the crosspiece 208 of the elongated lever arm 200 engages with the jut 62 of the canister unit 22. Particularly, the tapered second surface 212 of the crosspiece 208 in connection with a complementary slope or tapering of the jut 62 (cf. FIGS. 4 and 11), provides for the crosspiece 208 to slide on and slide past the jut 62 during the rotational movement. Simultaneously, and as visible in the view of FIG. 12B, continued rotation and engagement between the jut 62 and the crosspiece 208 forces and/or biases the elongated lever arm 200 radially outward, thereby releasing the third straight edge surface 214 from the stop provided on the lid unit (stop not visible) and allows the crosspiece 208 to slide past the jut 62 to the position illustrated in FIG. 12C (due to the rotational movement of the annular skirt 26 in the direction of arrow Q). FIG. 12C further illustrates how the thread 60 of the annular skirt 26 engages with and is positioned below the jut 62, whereby it can slide underneath the jut 62 upon rotation of the annular skirt 26 (cf. also FIG. 11). Thus, it is understood that the jut(s) 62 has at least a double functionality according to the disclosure. The arrangement of the elongated lever arm 200 and the elements cooperating therewith, according to the embodiments illustrated in FIGS. 12A-12C, inter alia provides the advantageous benefit that the annular skirt 26 is only rotatable when it is certain that the lid unit is correctly positioned on the canister unit 22.

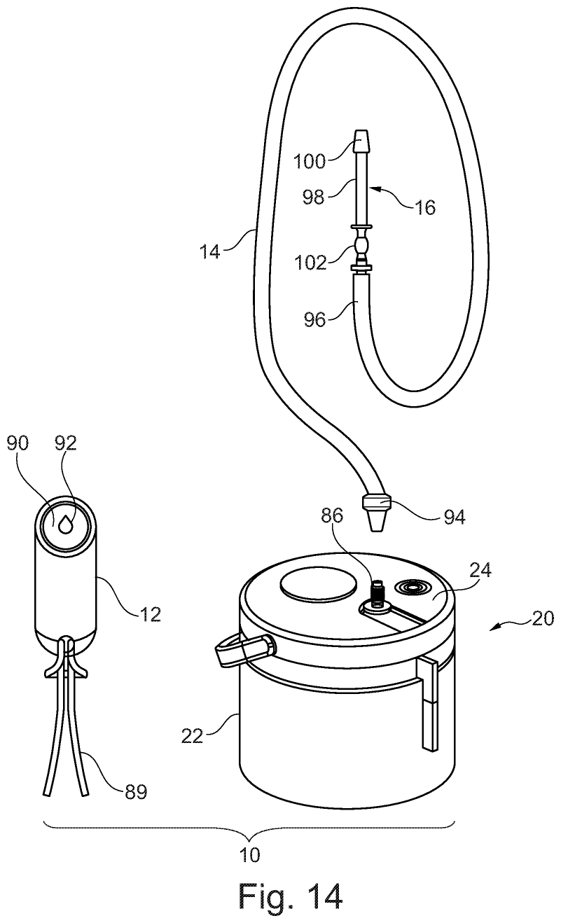

FIG. 14 is a schematic, perspective view illustrating components of one embodiment of a system 10 for irrigating the bowels of a user. In FIG. 14, the components of the system 10 are shown in a not fully assembled condition.

In FIG. 14, the system 10 includes a portable base 20 according to embodiments of the first aspect. The system 10 also includes a remote-control unit 12 adapted to communicate wirelessly with a control component (not shown) of the portable base 20 to control the irrigation system. In embodiments, either the remote-control unit 12 or the control component or both, includes a central processing unit (CPU). The remote-control unit 12 includes a cord or string 89 for attachment around the wrist of the user or similar. In the embodiment of FIG. 14, the remote-control unit 12 includes one button 90. The remote-control unit 12 and/or the button 90 can include indicia or pictograms 92 indicating one or more functions and/or instructions to a user of the system, such as, but not limited to, the possibility of release or flow of irrigation liquid from the canister unit 22 for commencement of a bowel irrigation cycle.

FIG. 14 illustrates one embodiment of a system including the canister unit 22 and the lid unit 24 of the portable base 22 disclosed herein, and further tubing 14, and a catheter or anal probe 16. A first end portion 94 of the tubing 14 is configured for connection and/or attachment with the connection 86 on the lid unit 24. A second end portion 96 of the tubing 14 is configured for connection and/or attachment with the anal probe 16. These connections assist in providing fluid communication between the irrigation liquid in the canister unit 22 and anal probe 16 via the tubing 14. The catheter or anal probe 16 is configured to be inserted into the rectum of a user. The anal probe 16 includes a catheter portion 98 for insertion into the rectum of the user to release irrigation liquid from the catheter into the bowels of the user. The catheter portion 98 includes a catheter tip 100. The anal probe 16 further includes a handle portion 102, which is connectable to the tubing 14. Although not illustrated in FIG. 14, the system 10 can optionally include a set of instructions for use, and/or a bag for storing the components of the system.

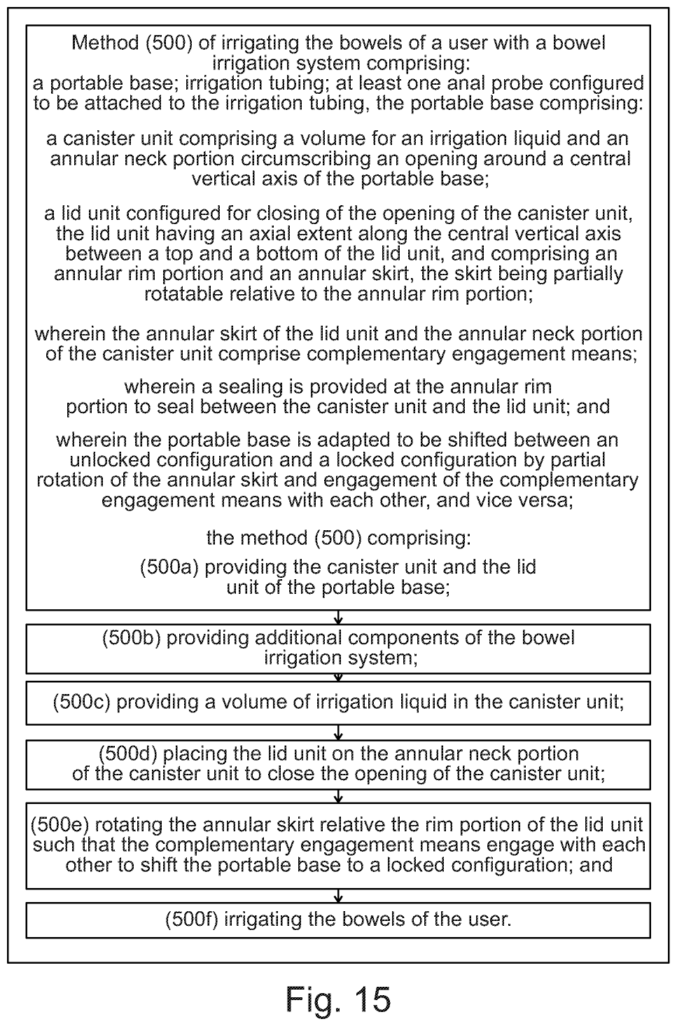

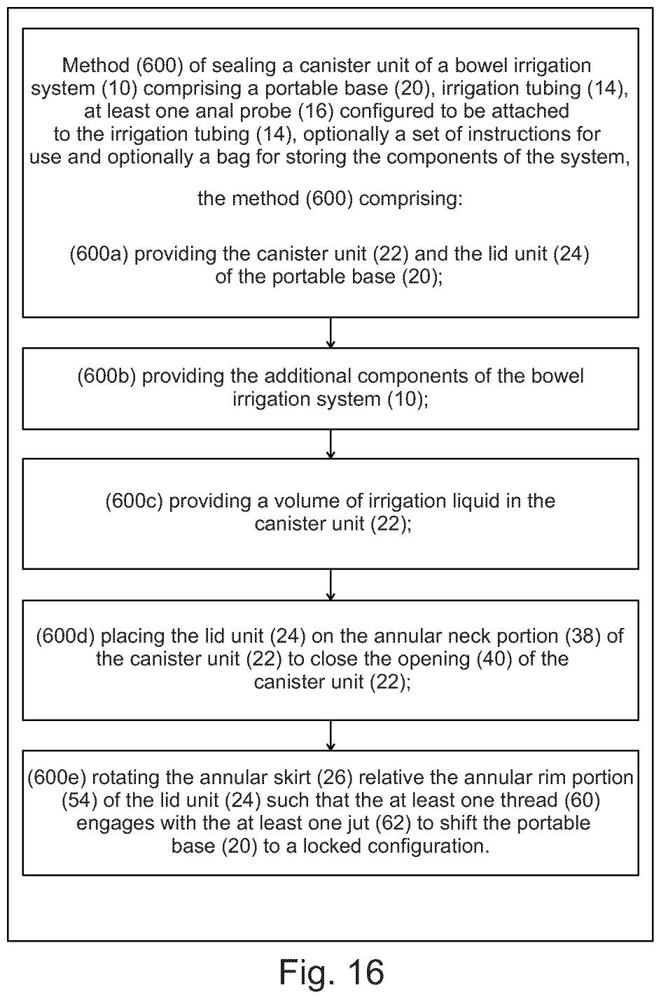

FIG. 15 is a diagram illustrating the steps (500a)-(500f) of a method (500) of irrigating the bowels of a user with a bowel irrigation system incorporating a portable base according to the first aspect.

Although particular features have been shown and described, it will be understood that they are not intended to limit the claimed invention, and it will be made obvious to those skilled in the art that various changes and modifications may be made without departing from the scope of the claimed invention. The specification and drawings are, accordingly, to be regarded in an illustrative rather than restrictive sense. The claimed invention is intended to cover all alternatives, modifications, and equivalents.

### Index of Reference Numbers

10 system for irrigating the bowels of a user
12 remote-control unit
14 tubing
16 anal probe or catheter
20 portable base
22 canister unit
23 tab or finger-hold (of canister unit)
24 lid unit
24a top of the lid unit
24b bottom of the lid unit
25 bottom portion of canister unit
26 annular skirt
27 gripping tab (of annular skirt)
28 lateral wall
30 sidewall of canister unit
32 cap (of lid unit)
34 handle
38 annular neck portion (of canister unit)
39 outward facing surface (of annular neck portion)
40 opening
42 vertical flange (of annular neck portion)
46 shoulder portion (of canister unit)
54 annular rim portion of lid unit
55 upper portion (of lateral wall)
56 radially outward facing surface of annular rim portion of lid unit
58 shoulder between annular rim portion and upper portion of lateral wall
59 radially inward facing surface of annular skirt
60 thread of annular skirt
61 radially outward facing surface of annular neck portion of canister unit
62 jut(s) on radially outward facing surface of annular neck portion
63 support flange
64 end portion of thread
66 vertical stop
72 sealing
76 through-going passage
78 volume of the canister unit
80 internal sealing of cap
84 internal chamber
86 connection for engagement of tubing
88 through-going passage
89 cord or string for remote-control
90 button
91 communication port
94 first end portion of tubing
96 second end portion of tubing
98 catheter portion
100 catheter tip
102 handle portion
104a, 104b sets of projections
106, 107, 108, 109 projections
110a, 110b sets of indentations
112, 114, 116, 118 indentations
174, 274 first portion of sealing
176. 276 second portion of sealing
178, 278 connecting portion of sealing
180, 280 radial inner surface of first portion of sealing
182, 282 protrusions
183, 283 abutment section
184, 284 lip section
186, 286 first flange
188 rounded-off end portion of flange
190, 290 downwardly facing surface of flange
191 upwardly facing surface of flange
192 second flange of lip section
194, 294 top surface of annular neck portion
196 edge of flange
198 screen
200 elongated lever arm
202 first end portion of elongated lever arm
204 connecting portion of elongated lever arm
206 second end portion of elongated lever arm
208 crosspiece of elongated lever arm
210 first surface of crosspiece
212 second surface of crosspiece
214 third surface of crosspiece
295 apex of transition between second portion and connecting portion
500 method of irrigating bowels
500a-500f method steps

1. A portable base for a system for irrigating the bowels of a user, comprising:
a canister unit comprising a volume for an irrigation liquid and an annular neck portion circumscribing an opening around a central vertical axis of the portable base; and
a lid unit configured to be positioned on the annular neck portion and over the opening for closing of the canister unit, the lid unit having an axial extent along the central vertical axis between a top and a bottom of the lid unit, and comprising an annular rim portion and an annular skirt, the annular skirt configured to be partially rotatable relative to the annular rim portion when the lid unit is positioned aright on the annular neck portion;
wherein a radially outward facing surface of the annular neck portion of the canister unit comprises at least one projecting jut;
wherein a radially inward facing surface of the annular skirt of the lid unit comprises at least one thread;
wherein a sealing is provided at the annular rim portion to seal between the canister unit and the lid unit in a locked configuration of the portable base;
wherein the sealing is movable in response to a force exerted by the annular neck portion; and
wherein rotating the annular skirt relative to the annular rim portion causes the at least one thread of the annular skirt to engage with the at least one jut of the annular neck portion and thereby move the sealing and lock the lid unit onto the canister unit in the locked configuration of the portable base.

2. The portable base of claim 1, wherein the sealing comprises at least a first portion fixed to the lid unit and a second portion configured to engage with the canister unit and a connecting portion extending between the first portion and the second portion.

3. The portable base of claim 2, wherein the first portion is fixed to the annular rim portion of the lid unit and the second portion is configured to engage with the annular neck portion of the canister unit, and wherein the force exerted by the annular neck portion is exerted on the second portion of the sealing.

4. The portable base of claim 2, wherein the second portion of the sealing comprises an abutment section configured for engagement with a top surface of the annular neck portion of the canister unit and a lip section configured to seal against one of a radially inward facing surface and a radially outward facing surface of the annular neck portion.

5. The portable base of claim 4, wherein the lip section is configured to seal against the radially inward facing surface of the annular neck portion.

6. The portable base of claim 4, wherein the abutment section comprises a first flange and the lip section comprises an apex of a transition between the second portion and the connecting portion of the sealing.

7. The portable base of claim 4, wherein the abutment section comprises a first flange and the lip section comprises a second flange and wherein the first flange and the second flange define an angle of at least 30° between each other.

8. The portable base of claim 7, wherein the first flange and the second flange are substantially perpendicular to each other.

9. The portable base of claim 1, wherein a radially inward facing surface of the annular neck portion of the canister unit comprises at least one set of pairwise mutually spaced projections, each projection having a circumferential length, and wherein a radially outward facing surface of the annular rim portion of the lid unit comprises at least one set of pairwise mutually spaced indentations that are complementary to the spaced projections.

10. The portable base of claim 9, wherein alignment of the pairwise mutually spaced indentations of the radially outward facing surface of the annular rim portion of the lid unit with the pairwise mutually spaced projections on the annular neck portion of the canister unit provides the aright positioning of the lid unit in relation to the canister unit.

11. The portable base of claim 1, wherein an elongated lever arm is attached to the radially inward facing surface of the annular skirt and is configured to engagingly cooperate with the at least one projecting jut provided on the radially outward facing surface of the neck portion of the canister unit.

12. The portable base of claim 11, wherein the elongated lever arm comprises a first end portion fixed to the radially inward facing surface of the annular skirt, a connecting portion extending from the first fixed end portion to a second end portion, the connecting portion and the second end portion being spaced from the radially inward facing surface of the annular skirt.

13. The portable base of claim 12, wherein the second end portion of the lever arm is formed as a crosspiece which is substantially transverse to the connecting portion.

14. The portable base of claim 1, wherein the annular skirt comprises a gripping tab projecting radially outward from the annular skirt.

15. The portable base of claim 14, wherein the canister unit comprises a finger-hold projecting laterally outward from a sidewall of the canister unit.

16. The portable base of claim 15, wherein the gripping tab of the annular skirt is configured to be aligned with the finger-hold of the canister unit when the portable base is placed in the locked configuration.

17. The portable base of claim 1, wherein when the lid unit is locked onto the canister unit, the portable base is configured to be pressurized and remain pressurized in the locked configuration.

18. The portable base of claim 1, wherein the opening of the canister unit has a diameter of between 100-150 mm.

19. The portable base of claim 1, wherein an upper portion of a lateral wall of the lid unit comprises a through-going passage.

20. The portable base of claim 19, wherein the annular skirt comprises a screen configured to cover the through-going passage of the lateral wall when the portable base is in the locked configuration.

21. A bowel irrigation system comprising the portable base of claim 1 and further comprising: irrigation tubing, at least one anal probe configured to be attached to the irrigation tubing, a remote control for controlling one or more control elements of the bowel irrigation system, a set of instructions for use, and a bag for storing the components of the bowel irrigation system.

22. Method of irrigating the bowels of a user with a bowel irrigation system of claim 21, comprising the steps of:
(a) providing the canister unit and the lid unit of the portable base;
(b) providing the additional components of a bowel irrigation system;
(c) providing a volume of irrigation liquid in the canister unit;
(d) placing the lid unit on the neck portion of the canister unit to close the opening of the canister unit;
(e) rotating the annular skirt on the rim portion of the lid unit such that the at least one thread of the annular skirt and the at least one jut of the canister unit engage with each other to shift the portable base to a locked configuration; and
(f) irrigating the bowels of the user.

23. Method of sealing a canister unit of a bowel irrigation system of claim 21, comprising the steps of:
(a) providing the canister unit and the lid unit of the portable base;
(b) providing the additional components of a bowel irrigation system;
(c) providing a volume of irrigation liquid in the canister unit;
(d) placing the lid unit on the neck portion of the canister unit to close the opening of the canister unit; and
(e) rotating the annular skirt on the rim portion of the lid unit such that the at least one thread of the annular skirt and the at least one jut of the canister unit engage with each other to shift the portable base to a locked configuration.
